SEQUENCE LISTING (98 sequences)

SEQ ID NO: 1 - DNA, 5305 bp, Homo Sapiens
gctctaggatgctgcggcaggttctgcacaggggcttgaggacgtgtttctcccggctcggccacttcattgccagtcaccctgtcttcttcgcctcggcgccggtgctcatctccatcctgctcggcgccagcttcagccgctaccaggtcgaggagagcgtggagcacctgctggcgccccagcacagcctggccaagatcgagcgcaacctcgttaacagcctcttcccggtcaaccgctccaagcaccgtctctactcggacctgcagacccccgggcgctacggccgggtcatcgtcacctccttccagaaagccaacatgctggaccagcatcacaccgacctgatcttaaagttgcatgctgctgtcaccaagatccaggttccaaggcctggttttaattacacgtttgcccatatatgtatcctgaataatgataagacttgcatcgtggatgacatagtgcacgtcctggaagagctaaagaatgctcgggccaccaatcggaccaattttgctatcacatacccaatcactcacttaaaggacgggagggctgtgtacaatgggcaccagcttgggggcgtcactgtgcacagcaaagaccgggtgaaatctgcagaggccatccagctcacctactacctgcagtcaatcaacagtctcaatgacatggtggctgagaggtgggagtccagcttctgcgacactgtcagactgtttcagaaatccaacagcaaagtcaaaatgtacccttacacgtcctcctcactgagggaagatttccagaagaccagccgcgtatcagaacgttacctggtcaccagcctgattctggtggttaccatggccatcctgtgttgctctatgcaggactgcgtccgcagcaaaccctggctaggcctgctcggattggtgaccataagcctggccactctcactgcagccgggatcatcaatcttactggtgggaaatataattccaccttcctgggagtccctttcgtcatgctaggtcatggattatatgggacttttgaaatgttatcctcctggaggaaaactagagaagaccaacatgttaaagagagaactgcagcagtctatgcagactccatgctctccttttctctcaccactgccatgtacctggtcacctttggcataggggccagccctttcacgaacattgaggcagccaggattttctgctgcaattcctgtattgcaatcttcttcaactacctctatgtactctcgttttatggttccagcctagtgttcactggctacatagaaaacaattaccagcatagtatcttctgtagaaaagtcccaaagcctgaggcattgcaggagaagccggcatggtacaggtttctcctgacggccagattcagtgaggacacagctgaaggcgaggaagcgaacacttacgagagtcacctattggtatgtttcctcaaacgctattactgtgactggataaccaacacctatgtcaagccttttgtagttctcttttaccttatttatatttcctttgccttaatgggctatctgcaggtcagtgaagggtcagaccttagtaacattgtagcaaccgcgacacaaaccattgagtacactactgcccagcaaaagtacttcagcaactacagtcctgtgattgggttttacatatatgagtctatagaatactggaacactagtgtccaagaagatgttctagaatacaccaaggggtttgtgcggatatcctggtttgagagctatttaaattaccttcggaaactcaatgtatccactggcttgcctaagaaaaatttcacagacatgttgaggaattcctttctgaaagcccctcaattttcacattttcaagaggacatcatcttctctaaaaaatacaatgatgaggtcgatgtagtggcctccagaatgtttttggtggccaagaccatggaaacaaacagagaagaactctatgatctcttggaaaccctgaggagactttctgtcacctccaaggtgaagttcatcgtcttcaatccgtcctttgtatacatggatcgatatgcctcctctctgggagcccccctgcacaactcctgcatcagtgctttgttcctgctcttcttctcggcattcctggtggcagattcactgattaacgtctggatcactctcacagttgtgtccgtggagtttggagtgataggtttcatgacattatggaaagtagaactggactgcatttctgtgctatgcttaatttatggaattaattacacaattgacaattgtgctccaatgttatccacatttgttctgggcaaggatttcacaagaactaaatgggtaaaaaatgccctggaagtgcatggggtagctattttacagagttacctctgctatattgttggtctgattcctcttgcagctgtgccttcaaatctgacctgtacactgttcaggtgcttgtttttaatagcatttgtcaccttctttcactgctttgccattttacctgtgatactgactttcctgccaccctctaagaaaaaaaggaaagagaagaaaaatcctgagaaccgggaggaaattgagtgtgtagaaatggtagatatcgatagtacccgtgtggttgaccaaattacaacagtgtgataatgtctgcttggcatattttcaccttaggtcttatcaagaccaaagagattatgttaatgaaacaattaaattcaaagttcttcccttttttaaagataggaaacaggcattgccaaaaaaaaaaaaaaaaaaaaaaggaaaggacagtggggagaaatgggcctggcatattttcagtctttaaaacaaaggagttgttatgagaattcacacacacatagacacacacacacacacacacacacacacacacacacacacaccctgggagacctatagtctcttaaactaagatcaagtagaagaaagcttattaacaagcaggatcctgccttatccaaactgcagatgttgctggcattgtgacaaaacccactgattgaaaggtcaactgccaaggcagaaacacctttaagcattgttcaaacaataaggcttccagaacttctgtagagcagtagctccagtcatggtctgtggtttgaggttttagctgtctcacctagctccctaacactgaaggagatacttgtgaaagttctgaccagcaaaagcaagccagagccttggaaactgatatgtggtagagtggccatcactcatggactaaaattgattcaccgctaaatttacccaggtgaagcagtttcgttgtctagaatgaaattatcatattccgccattggtatgcctttaacatttgtatagtttggtttgcttaaaacaccttaaaaccaatgacagctccagcactgcagaattggtgtgattctactttggaatagcttgtcacttgtcaccaaatgggtctgctttattagttacagctcttggcaggaggatccagggacccaaaaccacagggccaaacccaaatacctggcatgatggagcaaaagcaggtgtctacttggacccagatatagtgtctccattttaacaacaacaacaaaatagccagctggtacagctgtttgcattggccctacatgcattttttgcatggatatccagaaacatctgcccacacaaaactgcggggaaaaaaaatgaacactgaaatagttatttgctgttgcttccaacttgtagtgccagtctgcctttgctgtgaaacacacctgctcagagacagagaggggaagaagatctttggtaagtctaagtcctgacgctgagaagctttgtaaaagtgcagggagataaagggccaaaagggagatagatggaaaacactggaaaaagtattcactgatacaaatctatcaatgatggcagtccaattctcttgctaaagtggctgcacctcaccttgctggtcccccccacaccttttttgatgtccttctgcgtcatcatagcaaggcccttctgtaaattaacaagcctagatatttatactcttgacttccagtatctacagaagaatggttcatagatctaaacagaaatggtttagatctaaaaaggctgtatacgttgcccaggcccctgcatttctttaaatttataaaaatgaagctaaaacctggttacatttgaagcaaatatctacagtatttttcccttttagagatgtagcttccttagacatctgtagtggtaagcatttcccaaaagcatcttacctttctgaaccttagcagacatactgtgcagcttacctatcttctgcagaggaggaaactgagacctaggagaataaagtgactcactcaggtcacaccactaaagggttttcatcatttcagcatacctaagacagggcagtccaattttcagtattctcataagatggctattactcctctcaaaatgcatttccaaagtaggaacataggacttcgttggccacagggcagacatttttttagtgtctggaattaaaatgtttgaggtttaggtttgccattgtctttccaaaaggccaaataattcagatgtaaccacaccaagtgcaaacctgtgctttctatttcacgtactgttgtccatacagttctaaatacatgtgcaggggattgtagctaatgcattacacagtcgttcagtcttctctgcagacacactaagtgatcataccaacgtgttatacactcaactagaagataataagctttaatctgagggcaagtacagtcctgacaaaagggcaagtttgcataatagatcttcgatcaattctctctccaaggggcccgcaactaggctattattcataaaacacaactgaagaggggattggttttactgttaaatcatgtgttgctaaatcattttctgaacagtgtgttctaaatcagtcattgatttagtgtcagccacgtggagcacctcggcttaaagcagctccacaaaacctgacacaacacacacaccaattaaatggattttgttgagaatttaatcattcaatttggtcaaccagaatgacttcctgtggaactctgttttatgacagataatagttttccaacttgattgagtctctgtataccctgggatattgtattttttaatgaagggcattttcaaacttgtcaacttctcttttcagcacttgaaatgaaggcttatggaattctgactgtgaaatgaatttttctattgggaaaaaaaaaaaaaaa

SEQ ID NO: 2 - protein, 878 aa, Homo Sapiens
MLRQVLHRGLRTCFSRLGHFIASHPVFFASAPVLISILLGASFSRYQVEESVEHLLAPQHSLAKIERNLVNSLFPVNRSKHRKDLQTPGRYGRVIVTSFQKANMLDQHHTDLILKLHAAVTKIQVPRPGFNYTFAHICILNNDKTCIVDDIVHVLEELKNARATNRTNFAITYPITHLKDGRAVYNGHQLGGVTVHSKDRVKSAEAIQLTYYLQSINSLNDMVAERWESSFCDTVRLFQKSNSKVKMYPYTSSSLREDFQKTSRVSYLVTSLILVVTMAILCCSMQDCVRSKPWLGLLGLVTISLATLTAAGIINLTGGKYNSTFLGVPFVMLGHGGTFEMLSSWRKTREDQHVKERTAAVYADSMLSFSLTTAMYLVTFGIGDFTNIEAARIFCCNSCIAIFFNYLYVLSFYGSSLVFTGYIENNYQHSIFCRKVPKPEALQEKPAWYRFLLTARFSEDTAEGEEANTYESHLLVCFLKRYYCDWITNTYVKPFVVLFYLIYISFALMGYLQVSEGSDLSNIVATATQTIEYTTAQQKYFSNYSPVIGFYIYESIEYWNTSVQEDVLEYTKGFVRISWFESYLNYLRKLNVSTGLPKKNFTDMLRNSFLKAPQFSHFQEDIIFSKKYNDEVDVVASRMFLVAKTMNREELYDLLETLRRLSVTSKVKFIVFNPSFVYMDRYASSLGAPLHNSCISALFLLFFSAFLVADSLINVWITLTVVSVEFGVIGFMTLWKVELDCISVLCLIYGINYTIDNCAPMLSTFVLGKDFTRTKWVKNALEVHGVAILQSYLCYIVGLIPLAAVPSNLTCTLFRCLFLIAFVTFFHCFAILPVILTFLPPSKKKRKEKKNPENREEIECVEMVDIDSTRVVDQITTV

SEQ ID NO: 3 - protein, 10 aa, Homo Sapiens
KIERNLVNSL

SEQ ID NO: 4 - protein, 30 aa, Homo Sapiens
NFAITYPITHLKDGRAVYNGHQLGGVTVHS

SEQ ID NO: 5 - protein, 13 aa, Homo Sapiens
FLGVPFVMLGHGG

SEQ ID NO: 6 - protein, 11 aa, Homo Sapiens
TREDQHVKERT

SEQ ID NO: 7 - protein, 14 aa, Homo Sapiens
TAEGEEANTYESHL

SEQ ID NO: 8 - protein, 10 aa, mus musculus
KIERNLVNSL

SEQ ID NO: 9 - protein, 30 aa, mus musculus; identical to SEQ ID NO: 4

SEQ ID NO: 10 - protein, 13 aa, mus musculus
FLGVPFVMLGHGG

SEQ ID NO: 11 - protein, 11 aa, mus musculus
TREDQHVKERT

SEQ ID NO: 12 - protein, 14 aa, mus musculus
TAEGEEANTYESHL

SEQ ID NO: 13 - protein, 10 aa, Monodelphis domestica
KIERNLVNSL

SEQ ID NO: 14 - protein, 30 aa, Monodelphis domestica
NFAITYPITHLKDGREVYNGHQLGGVTVHS

SEQ ID NO: 15 - protein, 13 aa, Monodelphis domestica
FLGVPFVMLGHGG

SEQ ID NO: 16 - protein, 11 aa, Monodelphis domestica
TREDQHVKERT

SEQ ID NO: 17 - protein, 14 aa, Monodelphis domestica
TTDAEEANTYESHL

SEQ ID NO: 18 - protein, 5 aa, Gallus gallus
MVDVL

SEQ ID NO: 19 - protein, 30 aa, Gallus gallus; identical to SEQ ID NO: 14

SEQ ID NO: 20 - protein, 13 aa, Gallus gallus
FLGIPFVMLGHGG

SEQ ID NO: 21 - protein, 11 aa, Gallus gallus
TREDQHVKERT

SEQ ID NO: 22 - protein, 10 aa, Danio rerio
KIEGNLVDSL

SEQ ID NO: 23 - protein, 30 aa, Danio rerio
VPPLRYPITKLKDGREAYIGHQLGGVLASG

SEQ ID NO: 24 - protein, 15 aa, Danio rerio
YLGIPFVMLGHGLFG

SEQ ID NO: 25 - protein, 11 aa, Danio rerio
TREDQHVKERV

SEQ ID NO: 26 - protein, 14 aa, Danio rerio
TTDSEETNTYESHL

SEQ ID NO: 27 - protein, 10 aa, Ornithorhynchus anatinus
KIERSLAGSL

SEQ ID NO: 28 - protein, 31 aa, Ornithorhynchus anatinus
AGGQVNYPNAKLKDGRSSFIGHQLGGVLETP

SEQ ID NO: 29 - protein, 15 aa, Ornithorhynchus anatinus
LLGVPFFAMGHGTKG

SEQ ID NO: 30 - protein, 11 aa, Ornithorhynchus anatinus
TRETLPFKDRV

SEQ ID NO: 31 - protein, 14 aa, Ornithorhynchus anatinus
QTSHHETNPYQNHF

SEQ ID NO: 32 - protein, 10 aa, Sterechinus neumayeri
VGSNSIPVSL

SEQ ID NO: 33 - protein, 25 aa, Sterechinus neumayeri
FPFLPLPPFGRVFVGSQLGGVDLYP

SEQ ID NO: 34 - protein, 15 aa, Sterechinus neumayeri
VSLMPFLIIGVGVDN

SEQ ID NO: 35 - protein, 11 aa, Sterechinus neumayeri
LSIYLPVHERM

SEQ ID NO: 36 - protein, 14 aa, Sterechinus neumayeri
DSKCRKPEGHIIHP

SEQ ID NO: 37 - protein, 10 aa, Caenorhabditis elegans
RKELSQLDHL

SEQ ID NO: 38 - protein, 31 aa, Caenorhabditis elegans
IDEMTLSQISDAIQFDSGGMTHLLGGVTLDD

SEQ ID NO: 39 - protein, 15 aa, Caenorhabditis elegans
AYSMPFIVFSVGVDN

SEQ ID NO: 40 - protein, 11 aa, Caenorhabditis elegans
TSSTETLEHRM

SEQ ID NO: 41 - protein, 14 aa, Caenorhabditis elegans
IAAQGDRSFEKNTI

SEQ ID NO: 42 - protein, 9 aa, Homo Sapiens
WNEDKAAAI

SEQ ID NO: 43 - protein, 14 aa, Homo Sapiens
VSVIRVASGYLLML

SEQ ID NO: 44 - protein, 12 aa, Homo Sapiens
LEPPCTKWTLSS

SEQ ID NO: 45 - protein, 12 aa, Homo Sapiens
KPKAKVVVIFLF

SEQ ID NO: 46 - protein, 11 aa, Homo Sapiens
HRSFSNVKYVM

SEQ ID NO: 47 - protein, 8 aa, Homo Sapiens
QRLVDADG

SEQ ID NO: 48 - protein, 9 aa, mus musculus
WNEDKAAAI

SEQ ID NO: 49 - protein, 14 aa, mus musculus
VSVIRVASGYLLML

SEQ ID NO: 50 - protein, 12 aa, mus musculus
LEPPCTKWTLSS

SEQ ID NO: 51 - protein, 12 aa, mus musculus
KPKAKVVVILLF

SEQ ID NO: 52 - protein, 11 aa, mus musculus
HRSFSNVKYVM

SEQ ID NO: 53 - protein, 8 aa, mus musculus
QRLVDADG

SEQ ID NO: 54 - protein, 9 aa, Monodelphis domestica
WNEDKAAAI

SEQ ID NO: 55 - protein, 14 aa, Monodelphis domestica
VSVIRVASGYLLML

SEQ ID NO: 56 - protein, 12 aa, Monodelphis domestica
LEPPCTKWTLSS

SEQ ID NO: 57 - protein, 12 aa, Monodelphis domestica
KPKAKVVVILLF

SEQ ID NO: 58 - protein, 11 aa, Monodelphis domestica
HKSFSSVKYVM

SEQ ID NO: 59 - protein, 8 aa, Monodelphis domestica
QRLVDADG

SEQ ID NO: 60 - protein, 9 aa, Gallus gallus
WNEDKAAAI

SEQ ID NO: 61 - protein, 14 aa, Gallus gallus
VSVIRVASGYLLML

SEQ ID NO: 62 - protein, 12 aa, Gallus gallus
LEPPCTKWTLST

SEQ ID NO: 63 - protein, 12 aa, Gallus gallus
KPKAKVVVILLF

SEQ ID NO: 64 - protein, 11 aa, Gallus gallus
HRSFSNVTYVL

SEQ ID NO: 65 - protein, 8 aa, Gallus gallus
QRLVDADG

SEQ ID NO: 66 - protein, 9 aa, Xenopus laevis
WNEDKAAAI

SEQ ID NO: 67 - protein, 14 aa, Xenopus laevis
VSVIRVASGYLLML

SEQ ID NO: 68 - protein, 12 aa, Xenopus laevis
QCTPDSKWTLSS

SEQ ID NO: 69 - protein, 12 aa, Xenopus laevis
KPKTKVAVILGF

SEQ ID NO: 70 - protein, 11 aa, Xenopus laevis
HKSFVGVRYVL

SEQ ID NO: 71 - protein, 8 aa, Xenopus laevis
QRLVDADG

SEQ ID NO: 72 - protein, 9 aa, Danio rerio
WNEDKAAAI

SEQ ID NO: 73 - protein, 14 aa, Danio rerio
VSVIRVASGYLLML

SEQ ID NO: 74 - protein, 12 aa, Danio rerio
LDSPYSRWTFAS

SEQ ID NO: 75 - protein, 12 aa, Danio rerio
QSTTKVVVIFLF

SEQ ID NO: 76 - protein, 11 aa, Danio rerio
HQRFGSVKYIL

SEQ ID NO: 77 - protein, 8 aa, Danio rerio
QRLVSADG

SEQ ID NO: 78 - protein, 11 aa, Drosophila melanogaster
WTQEKAAEVLN

SEQ ID NO: 79 - protein, 14 aa, Drosophila melanogaster
PSALSIVIGVAVTV

SEQ ID NO: 80 - protein, 5 aa, Drosophila melanogaster
FSLAT

SEQ ID NO: 81 - protein, 12 aa, Drosophila melanogaster
RSWVKFLTVMGF

SEQ ID NO: 82 - protein, 11 aa, Drosophila melanogaster
HDSFVRVPHVI

SEQ ID NO: 83 - protein, 8 aa, Drosophila melanogaster
NRLVNSDG

SEQ ID NO: 84 - protein, 11 aa, Caenorhabditis elegans
WNETAAEQVLQ

SEQ ID NO: 85 - protein, 14 aa, Caenorhabditis elegans
FNYTIILAGYALML

SEQ ID NO: 86 - protein, 5 aa, Caenorhabditis elegans
WSLHS

SEQ ID NO: 87 - protein, 12 aa, Caenorhabditis elegans
KPASKVAIIVGC

SEQ ID NO: 88 - protein, 11 aa, Caenorhabditis elegans
RQSIGSSKYVI

SEQ ID NO: 89 - protein, 8 aa, Caenorhabditis elegans
IRLVDASG

SEQ ID NO: 90 - DNA, 736 bp, Homo Sapiens
  misc_feature 704: n = A,T,C or G
tctacacaaaccagatgaaccttccaatctcctgcctcgagtattgaagcctggctactgtgactgtggggaagggattaatggtctcagcattcagccaacaacaatacctgctcactataagcattcagaaaacagaaaagtttcaagaagcaggaagaaaagactcacctatgatcccaacacccagagataagagtcctgaagctcagatgacacagctgataacagggaagccaggacagaatctcattgttttgaacaccaaaacccgttcccttgacaacttggctatactacactattcgaatgttgcagatactgtggtcacatttcaaaggccagatctttcccagggcttaagctgttccttggatacttttggtaagtcatttatccactaatcatttagtaatcgtctctgacatgccaaacaccctgctcagggctggaaatgcagaacctgggaagccactggccttgtcctcaagatctctctctggctccctttgaatttgctaattcagactttcacatttcccccaggaaaaatcataaggaccaaatcatatccgttttctcaaatggcttcaaagacccatgtcatcgtttggcatcatgtaattctttactgatgtactttaagagtcacgttttattctctttatgcagctgtcaaggacagacacaaagagggggggggnggccttcctcactaaatacttttcccacaaca

SEQ ID NO: 91 - DNA, 557 bp, Homo Sapiens
acaactgcagcgagagaagaggctggcagcatgggtggcaggaggcttggcagcctcacaggatgcctgcaaatacctttcacttatgcagtttggcagtgcagtggtgcatggagacagcgtcttgggcctggcacccacagtcacttaggaagttggagtcctggagaggagaacacagaacgtggacaactagctgtcaaattgcagtaaaagttggttcaggaaaagtggaagctacccagatgtctaccaattgagatgaaccatccaatctcctgcctcgagtattgaagcctggctactgtgactgtggggaagggattaatggtctcagcattcaaagcttctattctggaatagaacagctagcatactaccaagacttttcaaggagcaagaatggagctccctggagaactgactgaacatggcttcagaggcagtatccatgtcacatttcaaaggccagatctttcccagggcttaagctgttccttggatacttttgctgatggtttacacatcttcttcccacattatattgtaactttctt

SEQ ID NO: 92 - DNA, 601 bp, Homo Sapiens
atttttaaaaaatatgctgaatttgaagtttctttcaaagtacagtgtttcaatggggggagtccaatttttgtaaaattttacaaaaactgtattgccctaaaggcagcctactgcacacaaggatcacagtgacttttacttgttattctacatgattacttaaaatttttctgatttttttaccctcatctatcttctaacttgtctagttaactcttaagaatttcaaattttctttgaaagatgataggcaatatgagatgagagataatctacaaaagttacagatgctcacatgtataaaacagtcaaaatatcacaggtcaatgacataaactgcattaaataaattatgtttataggcatcagtagttgaaaatgctcaataattctgggctccttccccaaaatgtaagacttaagtacttcaaaggcattattctttactcatgaggatcagtggcttcatttagtaaaagaaaaaggaatggacccaggatcccagtaaataattactaactgatcgcaacgctcttttatctaatgaacaaccaacaaccaacagaaaacccttgattcacagaggagcaagtcctag

SEQ ID NO: 93 - DNA, 126 bp, Homo Sapiens
caattggtagacatctgggtagcttccacttttcctgaaccaacttttactgcaatttgacagctagttgtccacgttctgtgttctcctctccaggactccaacttcctaagtggctgtgggtgc

SEQ ID NO: 94 - DNA, 455 bp, Homo Sapiens
acctgtgcgtggccgttcccgccgccgccgcaggtctatcccggggccgaagccggcgcccgccttctcggggaattctccggagggggagtgcgaggggaaccacggtgactgcctgctagctcacggctggcgcgcacacgcacacgcccaactttgccaagccgtcggcgccccgcgggctcccccgcgccccctgcggctcaacacgctcggagacctgtatctctcctgctctgagataaggttccctccactctcacaccttcgcatgtaggggaggagagggcggagtgaggcagagaagggggttaatgctactgactccctggccagcctttctcaaacactctacgcccgcaggggcgcccgcgccagccacgccgcaccaggtcccccagacctgctggtgacgacagagagaggaggaggaagagaaggcagggcgaagaacc

SEQ ID NO: 95 - DNA, 43 bp, Homo Sapiens
cttttgagtggacgtgctccagacacacacccggaccccgtgg

SEQ ID NO: 96 - DNA, 736 bp, Homo Sapiens
  misc_feature 22, 704, 707: n = A,T,C or G
tctacacaaaccagatgaaccntccaatctcctgcctcgagtattgaagcctggctactgtgactgtggggaagggattaatggtctcagcattcagccaacaacaatacctgctcactataagcattcagaaaacagaaaagtttcaagaagcaggaagaaaagactcacctatgatcccaacacccagagataagagtcctgaagctcagatgacacagctgataacagggaagccaggacagaatctcattgttttgaacaccaaaacccgttcccttgacaacttggctatactacactattcgaatgttgcagatactgtggtcacatttcaaaggccagatctttcccagggcttaagctgttccttggatacttttggtaagtcatttatccactaatcatttagtaatcgtctctgacatgccaaacaccctgctcagggctggaaatgcagaacctgggaagccactggccttgtcctcaagatctctctctggctccctttgaatttgctaattcagactttcacatttcccccaggaaaaatcataaggaccaaatcatatccgttttctcaaatggcttcaaagacccatgtcatcgtttggcatcatgtaattctttactgatgtactttaagagtcacgttttattctctttatgcagctgtcaaggacagacacaaagagggggggggnggncttcctcactaaatacttttcccacaaca

SEQ ID NO: 97 - DNA, 601 bp, Homo Sapiens
aatgatgaatttatcctgacaaagtactgtattcactccaaaagaaatttaccaaaataaatgaacacacgaatatataaataaatagttttactttaaatgcattatttttttctcttagggaaataactggcttatataaaggacaatgtgtatatggtgtgtatgtttaaggcgtgcttcaaggttgctctcaagctgagccagaactatcacgagaagagtgaaaggagcacccgggacgcagaagttaaggaggcagttactcctagggtcctgtaagtgctggcagggtcagcccgtgagagtgagtgcctctttaaatttgcgtcacagacgcctgcttacctcaccccagtccaagccctgtgattggtcaggccatcaaagcctcgccccctacacgacccggaattcgacgccaacactggtttctggggcaacttctgcgtagctatgtgactagcacccggaaataattgccaccgccatcttttggtgcagaaggtgacgggaaacaggccgcagacctgaacttccaaccgtatgtaggcgagaagccggtgccgatactcccactatcccacaatgtcccactggg

SEQ ID NO: 98 - DNA, 601 bp, Homo Sapiens; identical to SEQ ID NO: 92

(19) United States
(12) Patent Application Publication
(10) Pub. No.: US 2012/0100995 A1
(43) Pub. Date: Apr. 26, 2012

(54) Biomarkers for Autism Spectrum Disorders

(75) Inventors: Stephen W. Scherer, Toronto (CA); John B. Vincent, Toronto (CA)

(73) Assignee: The Hospital for Sick Children

(21) Appl. No.: 13/231,467

(22) Filed: Sep. 13, 2011

Related U.S. Application Data
(60) Provisional application No. 61/382,834, filed on Sep. 14, 2010.

(30) Foreign Application Priority Data
Jun. 9, 2011 (CA) 2,744,424

(51) Int. Cl. C12Q 1/68; C40B 20/00

(52) U.S. Cl. 506/2; 435/6.12; 435/6.11

(57) ABSTRACT

Methods of determining the risk of ASD or ID in an individual are provided which comprise identifying the presence of one or more specific genomic mutations in, upstream of, or comprising the PTCHD1 gene. Additionally provided are methods of determining the risk of ASD or ID in an individual comprising analyzing genomic mutations in PTCHD1AS1 and/or PTCHD1AS2 and/or PTCHD1AS3.

### RELATED APPLICATIONS

This application claims the benefit of U.S. Provisional Application No. 61/382,834, filed on Sep. 14, 2010. This application claims priority under 35 U.S.C. §119 or 365 to Canadian Application No. 2,744,424, filed Jun. 9, 2011.

The entire teachings of the above applications are incorporated herein by reference.

### FIELD OF THE INVENTION

The present invention relates to genetic markers for Autism Spectrum Disorders (ASD), and methods of determining risk of ASD in an individual.

### BACKGROUND OF THE INVENTION

Autism (MIM 209850) is a severe, lifelong neurodevelopmental disorder characterized by impairments in communication and socialization, and by repetitive behavior. Autism is not a distinct categorical disorder but is the prototype of a group of conditions defined as Pervasive Developmental Disorders (PDDs) or Autism Spectrum Disorders (ASD), which include Asperger's Disorder, Childhood Disintegrative Disorder, Pervasive developmental disorder-not otherwise specified (PDD-NOS) and Rett Syndrome. ASD is diagnosed in families of all racial, ethnic and social-economic backgrounds with incidence roughly four times higher in males compared to females. Data from several epidemiological twin and family studies provide substantial evidence that autism has a significant and complex genetic etiology. The concordance rate in monozygotic twins is 60-90%, and the recurrence rate in siblings of affected probands has been reported to be between 5-10% representing a 50 fold increase in risk compared to the general population. Although autism spectrum disorders are among the most heritable complex disorders, the genetic risk is clearly not conferred in simple Mendelian fashion.

Recent studies of sub-microscopic genomic copy number variation (CNV) have identified several loci associated with Autism Spectrum Disorder (ASD; MIM 209850). De novo CNVs associated with ASD have been reported in ˜7% of simplex families and ˜2% of multiplex families. CNV studies have also led to the identification of autism candidate genes such as SHANK3 (MIM 606230) and NRXN1 (MIM 600565). Intellectual disability (ID) is frequently associated with autism (in up to ˜30% of cases for ASD, and ˜67% for autism). Moreover, mutations in several X-linked ID (XLID) genes (e.g. NLGN4 and IL1RAPL1) have been shown to result in an autistic phenotype, which suggests that autism and ID may often share a common genetic etiology. Currently available data suggest substantial genetic heterogeneity, with the most likely cause of non-syndromic idiopathic ASD involving multiple epistatically-interacting loci. The identification of large scale copy number variants (CNVs) represents a considerable source of genetic variation in the human genome that contributes to phenotypic variation and disease susceptibility found in small inherited deletions in autistic kindreds, suggesting possible susceptibility loci.

It would thus be desirable to characterize putative susceptibility loci to identify genetic markers of ASD, as well as to understand the role of candidate genes for ASD in order to facilitate determination of the risk of ASD in an individual, and to assist in the diagnosis of ASD.

### SUMMARY OF THE INVENTION

Systematic screening at PTCHD1 and 5′-flanking regions, suggests involvement of this locus in ˜1% of autism spectrum disorder (ASD) and intellectual disability (ID) individuals. Provided herein are mutations in the X-chromosome PTCHD1 (patched-related) locus, which are useful in assessing the risk of ASD and/or the risk of ID in an individual, as well as being useful to diagnose carrier status of an individual, or other condition(s). Provided markers are useful both individually and in the form of a microarray to screen individuals for risk of ASD and/or ID or for carrier status for risk of ASD and/or ID.

Thus, in one aspect of the present invention, a method of determining the risk of ASD in an individual is provided, comprising analyzing a nucleic acid-containing sample obtained from the individual for the presence or absence of a genomic sequence mutation at the PTCHD1 locus, wherein the mutation comprises a deletion of a region upstream to the PTCHD1 gene (e.g., a deletion as set forth in Table 2), a disruption of a non-coding RNA (ncRNA) selected from PTCHD1AS1, PTCHD1AS2, or PTCHD1AS3, or splice variants of these ncRNAs, or a disruption of other regulatory elements upstream of the PTCHD1 coding region. Presence of the mutations has been found to be indicative of ASD.

These and other aspects of the present invention are described by reference to the following figures.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 depicts the cDNA sequence (SEQ ID No:1) of a PTCHD1 (A) and the amino acid sequence (SEQ ID No: 2) of the protein it encodes (B).

FIG. 2 depicts detailed genomic organization of the PTCHD1 locus.

FIG. 3 depicts pedigrees of families. (A) Pedigrees showing PTCHD1 mutations. (B) Pedigrees showing deletions at the PTCHD1/PTCHD1AS1-3 locus.

FIG. 4 depicts PTCHD1 missense variants. Electropherograms indicate the nucleotide substitutions within PTCHD1 in unrelated ASD families and ID families.

FIG. 5 depicts PTCHD1 domain structure (A) and protein sequence conservation (B).

FIG. 6 depicts the consensus sequence for non-coding RNA of PTCHD1AS1 (SEQ ID No:11).

FIG. 7 depicts the consensus sequence for non-coding RNA of PTCHD1AS2 (SEQ ID No:12).

FIG. 8 depicts the consensus sequence for non-coding RNA of PTCHD1AS3 (SEQ ID No:13).

### DETAILED DESCRIPTION OF THE INVENTION

A method of determining the risk of an autism spectrum disorder (ASD) in an individual, or carrier status of an individual, is provided comprising screening a biological sample obtained from the individual for a mutation that may modulate the expression of PTCHD1.

The term “an autism spectrum disorder” or “an ASD” is used herein to refer to at least one condition that results in developmental delay of an individual such as autism, Asperger's Disorder, Childhood Disintegrative Disorder, Pervasive Developmental Disorder-Not Otherwise Specified (PDD-NOS) and Rett Syndrome (APA DSM-IV 2000).

The term “intellectual disability” or “ID” refers to a disability originating before age 18, characterized by significant limitations in both intellectual functioning and adaptive behavior as expressed in conceptual, social, and practical adaptive skills.

Microdeletions that directly disrupt the PTCHD1 gene have been identified in males in families affected with ASD, ID or learning disability. Identified deletions are maternally inherited and were not observed in more than 10,000 controls, indicating that these alterations are associated with ASD and ID. Maternally inherited missense mutations in PTCHD1 in male probands have also been reported.

PTCHD1 encodes a Patched-related protein with 12 transmembrane domains and a sterol-sensing domain, structurally similar to the Hh receptors PTCH1 and PTCH2, as well as the Niemann-Pick Type C1 protein (NPC1) and several others. Many Patched-related genes have been found in various organisms, from nematodes to humans, and they appear to play diverse biological functions, including cytokinesis, growth and pattern formation (Zugasti, O. et al., Genome Res. 15, 1402-1410 (2005)). For instance, there are just seven patched-related genes in humans (PTCH1, PTCH2, PTCHD1, PTCHD2, PTCHD3, NPC1 and c6orf138, whereas in C. elegans there are at least 26 patched-related genes, with diverse roles in development in addition to Hh signaling, including cytokinesis, growth and pattern formation (Zugasti, O. et al., Genome Res. 15, 1402-1410 (2005)). We have found in 10T1/2 cells, an inhibitory effect of PTCHD1 was demonstrated on Gli-dependent transcription. Although these results suggest that PTCHD1 exhibits biochemical activity in Hh-dependent processes similar to that of PTCH1 and 2, other functions or roles for PTCHD1 cannot be excluded at this point.

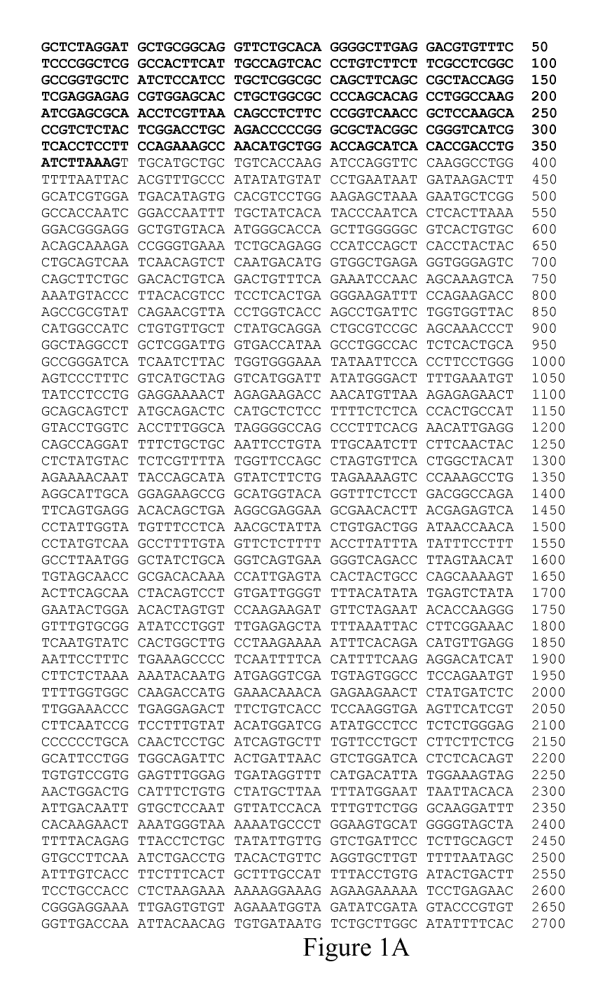

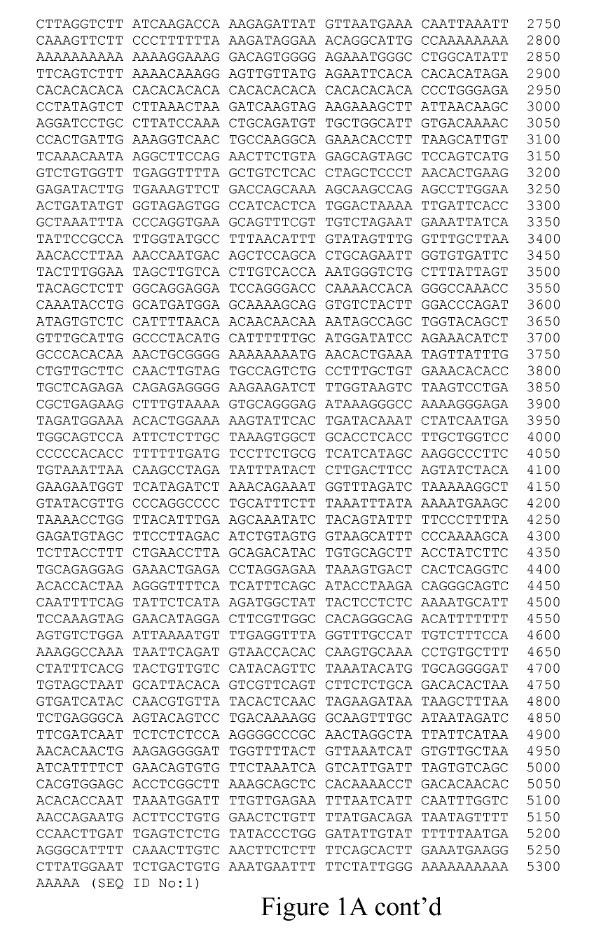

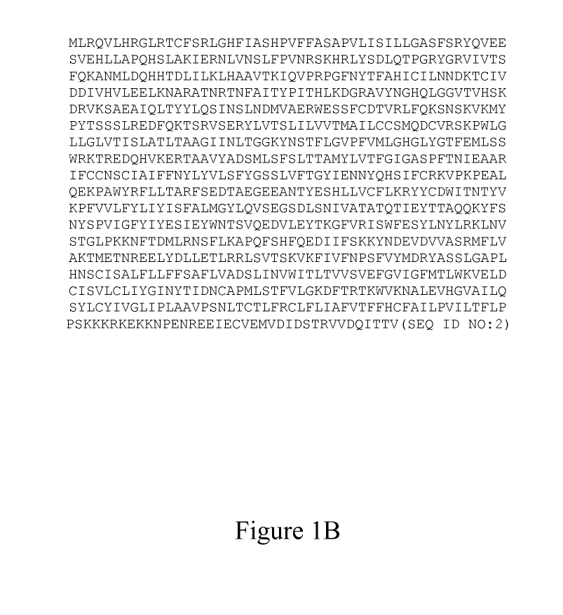

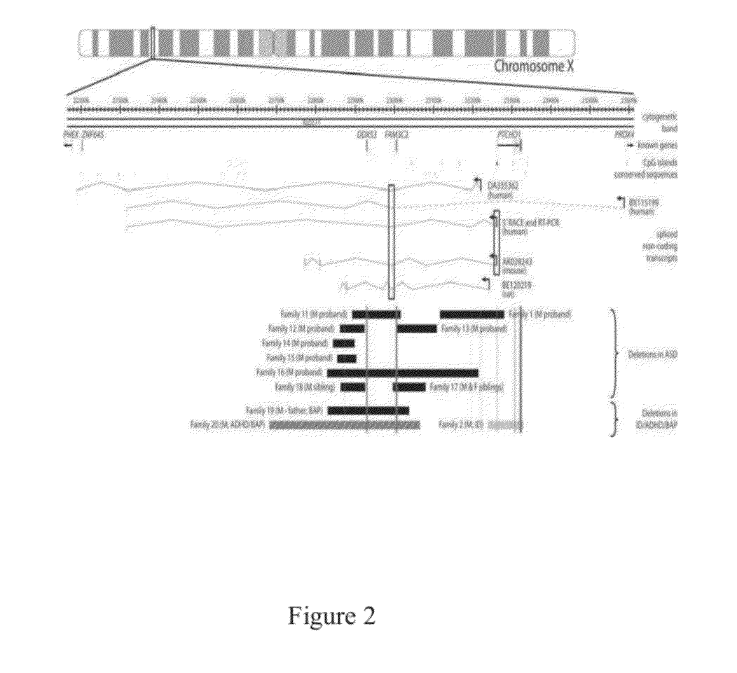

We have further characterized the PTCHD1 locus and found variants identified in PTCHD1 were not seen in more than 500 controls, further supporting a role of PTCHD1 in autism and ID. As used herein, the term “PTCHD1 locus” refers to the region in the X chromosome which extends from about the distal-most exon of mRNA clone DA355362 at the distal end to a proximal boundary which at least includes the coordinate according to the UCSC 2006/hg18 build ChrX:23,329,120 and which may extend to BX115199 as illustrated in FIG. 2. As will be appreciated by one of skill in the art, the PTCHD1 locus may encompass PTCHD1 corresponding to FIG. 1 or isoforms thereof.

Furthermore, 10 deletions were found that map to regions upstream of the coding region of PTCHD1. The region 5′ and distal to PTCHD1 is relatively gene poor. Within this upstream region, a coding gene, DDX53, encoding DEAD Box 53, lies ˜335 Kb 5′ to PTCHD1. Five of the 10 upstream deletions span DDX53. However, based on the function of the DDX53 protein and the expression pattern of this gene (which is restricted mainly to testis and tumor cells (Cho, B. et al., Biochem. Biophys. Res. Commun. 292, 715-726 (2002)), it is unlikely to contribute to the ASD or ID phenotype. Additionally, within the gene-poor region between PTCHD1 and DDX53, there is a putative pseudogene of FAM3C, FAM3C2, which is disrupted by five of the 10 upstream deletions. FAM3C, a cytokine-like gene on 7q31.31, consists of 10 exons (Zhu, Y. et al., Genomics 80, 144-150 (2002)) whereas FAM3C2, although 99% identical, has no intron/exon structure and is interrupted by a short interspersed nuclear element (SINE). It appears to have inserted on Xp22 after human/chimp evolutionary divergence. Since no mRNA or EST matches exactly to FAM3C2, it is most likely an untranscribed processed pseudogene.

The region just distal to PTCHD1 was examined in detail and a number of putative enhancer and promoter sequences were identified, as well as conserved (and putative regulatory) elements (FIG. 2). Several overlapping spliced long (>200 nt) non-coding (n c) RNAs (PTCHD1AS1 (from cDNA clone IMAGE:1560626; BX115199) and PTCHD1AS2 (from cDNA clone BRSTN2000219; DA355362)), were identified, which map to the opposite strand and distal to PTCHD1 (see FIG. 2). 5′RACE (Rapid Amplification of cDNA Ends) shows that a number of splice variants of these transcripts originate at the CpG island just upstream of PTCHD1, encompassing its putative promoter. Similar antisense transcripts are present at syntenic loci in other mammalian species, at least two exons of which appear to be conserved between rat, mouse and humans (see FIG. 2).

Although the ncRNAs do not appear to encode protein, they may serve as regulators for other coding genes, particularly for PTCHD1, since the 5′ exons are adjacent on opposite strands. Such ncRNAs may regulate expression of a coding transcript on the opposite strand through a number of mechanisms, including modification of chromatin, transcriptional regulation and post-transcriptional modification (Mercer, T. R. et al., Nat. Rev. Genet. 10, 155-159 (2009); Kleinjan, D. A et al., Am. J. Hum. Genet. 76, 8-32 (2005)).

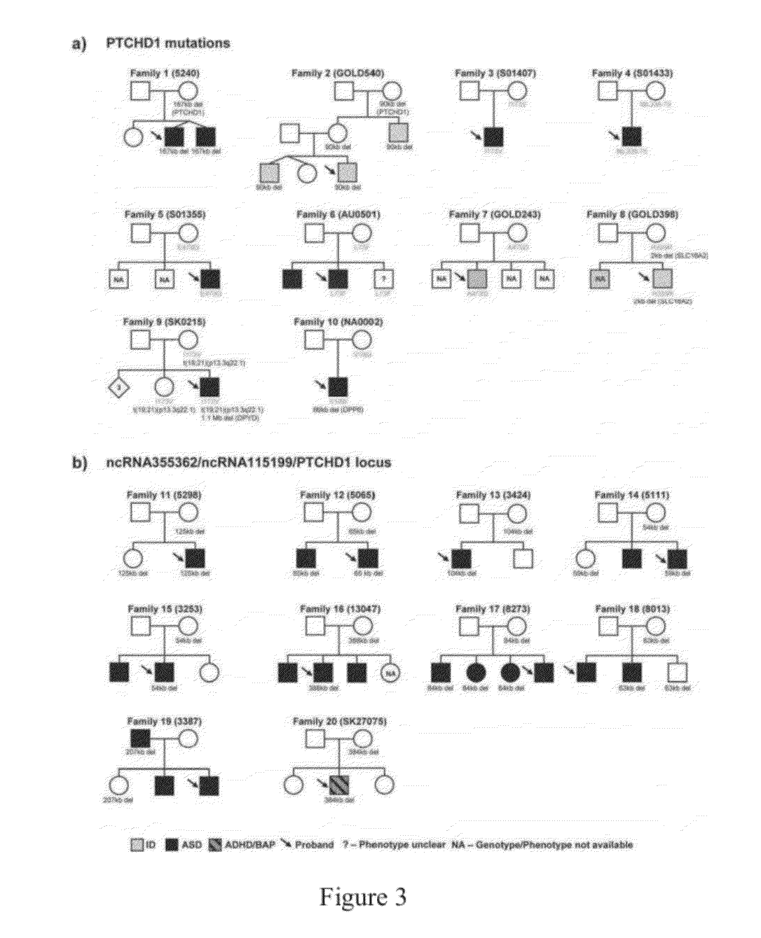

All of the upstream deletions identified, as well as PTCHD1 deletions (e.g., Family 1) disrupt conserved (and putative regulatory) sequences and/or exons of ncRNAs (see FIG. 2). Deletions were not inherited by a subset of the affected family members; also, missense variants do not segregate with disease in all families (e.g., Family 6) (FIG. 3). These findings are similar to other previously reported major affect ASD loci such as 16p11.2 (Weiss, L. A. et al., N. Engl. J. Med. 358, 667-675 (2008)) and are also consistent with the complex, non-Mendelian inheritance believed to control the etiology of autism. A recently proposed threshold model of relative contribution in ASD has been described (Cook, Jr., E. H. et al., Nature 455, 919-923 (2008).), whereby it is anticipated that multiple common and rare variants may act in concert to generate the phenotype. For instance, under this model, some de novo CNVs may be solely sufficient to cause ASD. Conversely, other de novo CNVs may have weaker effects, requiring contributions from additional loci (for example additional risk haplotypes, or other CNVs), or environmental risk factors, for the burden of contributory factors to cross a risk threshold and result in an ASD phenotype. In families that carry putative PTCHD1 missense mutations (e.g., Families 9 and 10), other CNVs involving genes that may also contribute to the phenotype were identified. In Family 9, in addition to the I173V substitution, a de novo ˜1.1 Mb loss was found at 1p21.3 resulting in deletion of the entire DPYD gene (MIM 274270), encoding dihydropyrimidine dehydrogenase (DPD) (Marshall, C. R. et al., Am. J. Hum. Genet. 82, 477-488 (2008)). Complete DPD deficiency results in highly variable clinical outcomes, with convulsive disorders, motor retardation, and mental retardation being the most frequent manifestations, and autistic features occasionally reported (van Kuilenburg, A. B. et al., Hum. Genet. 104, 1-9 (1999)). In this family, a balanced translocation, t(19; 21)(p13.2; q22.12) is also present in the proband, but is inherited from the unaffected mother and shared with an unaffected sister. In Family 10, which shows the V1951 substitution in PTCHD1, a 66 Kb de novo loss at 7q36.2 was previously reported that results in deletion of the third exon of DPP6 (MIM 126141)—previously reported as a positional and functional candidate gene for autism (Marshall, C. R. et al., Am. J. Hum. Genet. 82, 477-488 (2008)).

Thus, in ASD individuals there is evidence for the possible involvement of more than one locus in the disease, and these findings may support the threshold model of relative contribution in ASD and polygenic inheritance in autism. As such, some de novo CNVs may be highly penetrant in causing ASD susceptibility (e.g. disruption of PTCHD1 in Family 1). Conversely, other de novo CNVs (e.g. DPP6 and DPYD deletions) may have more subtle effects, requiring contributions of additional loci (e.g. PTCHD1 missense mutations in the case of Families 9 & 10) for ASD to be phenotypically evident. This scenario may also apply to the ID families with PTCHD1 mutations.

Cerebellar abnormalities have frequently been linked to autism, including recent magnetic resonance imaging (MRI) studies showing significant decrease in cerebellar grey matter (Courchesne, E. et al., Neurology 57, 245-254 (2001); Toal, F. et al., Br. J. Psychiatry 194, 418-425 (2009)), and decreased cerebellar connectivity and activity (Mostofsky, S. H. et al., Brain 132, 2413-2425 (2009)).

In the present methods, it is possible to determine ASD risk in an individual, as well as to determine carrier status of an individual (e.g., testing of females for the presence of mutations associated with ASD, to determine whether they are carriers). In the methods, a biological sample obtained from the individual is utilized. A suitable biological sample may include, for example, a nucleic acid-containing sample or a protein-containing sample. Examples of suitable biological samples include saliva, urine, semen, other bodily fluids or secretions, epithelial cells, cheek cells, hair and the like. Although such non-invasively obtained biological samples are preferred for use in the present method, one of skill in the art will appreciate that invasively-obtained biological samples, may also be used in the method, including for example, blood, serum, bone marrow, cerebrospinal fluid (CSF) and tissue biopsies such as tissue from the cerebellum, spinal cord, prostate, stomach, uterus, small intestine and mammary gland samples. Techniques for the invasive process of obtaining such samples are known to those of skill in the art. The present method may also be utilized in prenatal testing for the risk of ASD using an appropriate biological sample such as amniotic fluid and chorionic villus.

In one aspect, the biological sample is screened for nucleic acid encoding selected genes in order to detect mutations associated with an ASD. It may be necessary, or preferable, to extract the nucleic acid from the biological sample prior to screening the sample. Methods of nucleic acid extraction are well-known to those of skill in the art and include chemical extraction techniques utilizing phenol-chloroform (Sambrook et al., 1989), guanidine-containing solutions, or CTAB-containing buffers. As well, as a matter of convenience, commercial DNA extraction kits are also widely available from laboratory reagent supply companies, including for example, the QIAamp DNA Blood Minikit available from QIAGEN (Chatsworth, Calif.), or the Extract-N-Amp blood kit available from Sigma (St. Louis, Mo.).

Once an appropriate nucleic acid sample is obtained, it is subjected to well-established methods of screening, such as those described in the specific examples that follow, to detect genetic mutations indicative of ASD, i.e. ASD-linked mutations. Representative methods of screening include straight sequencing; use of arrays as described herein; as well as quantitative PCR (qPCR) and multiplex ligation-dependent probe amplification (MLPA). For example, various platforms can be used: affymetrix 500 k SNP arrays; Illumina 1M BeadChips; NimbleGen 385K arrays; Affymetrix 6.0 arrays; Illumina 550× arrays; and other platforms.

Mutations, including sequence mutations in coding and/or regulatory regions of a gene, as well as in flanking regions of a gene, have been found to be indicative of ASC. Representative mutations include, for example, genomic copy number variations (CNVs), which include gains and deletions of segments of DNA (e.g., segments of DNA greater than about 1 kb, such as DNA segments over about 50 kb, such as between 50 and 300 kb, or between about 300 and 500 kb); as well as base pair mutations such as nonsense, missense and splice site mutations.

Genomic sequence variations of various types in different genes have been identified as indicative of ASD. As described herein, deletions in the 5′ flanking region of PTCHD1 that disrupted a complex non-coding RNA (e.g., PTCHD1AS1, PTCHD1AS2, PTCHD1AS3), and potential regulatory element(s) in the PTCHD1 locus have been associated with ASD. In one embodiment, genomic sequence variations that alter the expression of PTCHD1 have been linked to ASD. The terminology “alter expression” refers broadly to sequence variations that may alter (e.g., inhibit, or at least reduce) any one of transcription and/or translation of the coding nucleic acid sequence of PTCHD1, as well as the activity of the PTCHD1 protein.

Genomic sequence variations other than CNVs have also been found to be indicative of ASD, including, for example, missense mutations which result in amino acid changes in a protein that may also affect protein expression. In one embodiment, missense mutations in the PTCHD1 gene have been identified which are indicative of ASD. In certain embodiments, a missense change is associated with a further genetic mutation and the presence of the combination of the missense change and the deletion is associated with ASD.

In another embodiment, sequence variations associated with ASD include deletions in the region that is within the 5′ region upstream of the PTCHD1 gene (e.g., in whole or in part, or a portion or more of the upstream region thereof). In certain embodiments, mutations include deletions (e.g., deletions described in Table 2). The term “upstream region,” as used herein, refers to a region that is distal to the PTCHD1 gene within approximately 1.2 mbp. For example, in one embodiment, the region comprises cDNA clone BRSTN2000219 (DA355362) (see FIG. 2). In another embodiment, the region comprises the 5′ RACE and RT-PCR region as shown in FIG. 2. In additional embodiments, the region comprises any of the regions comprising non-coding mRNA regions of PTCHD1AS1, PTCHD1AS2, and/or PTCHD1AS3 or splice variants thereof. Upstream regions can be of varying sizes, from under 1 kbp to over 1 mbp. Representative upstream regions include regions varying in size from approximately 50 kbp and approximately 1 mbp; from approximately 60 kbp and approximately 500 kbp; from approximately 100 kbp and approximately 400 kbp; from approximately 100 kbp to 300 kbp. In certain embodiments, representative upstream regions comprise one or more of the breakpoint deletions, for example, those identified in Table 2. In certain embodiments, representative upstream regions comprise chrX:22,200,000-23,260,000, chrX:22,300,000-23,260,000, chrX:22,670,000-23,260,000, chrX:22,900,000-23,260,000 or chrX:22,900,000-23,050,000.

To determine risk of ASD in an individual, it may be advantageous to screen for multiple genomic mutations, including CNVs and/or mutations as indicated above applying array technology. In this regard, genomic sequencing and profiling, using well-established techniques as exemplified herein in the specific examples, may be conducted for an individual to be assessed with respect to ASD risk/diagnosis using a suitable biological sample obtained from the individual. Identification of one or more mutations associated with ASD would be indicative of a risk of ASD, or may be indicative of a diagnosis of ASD. This analysis may be conducted in combination with an evaluation of other characteristics of the individual being assessed, including for example, phenotypic characteristics.

In view of the determination of gene mutations which are linked to ASD, a method for determining risk of ASD in an individual is also provided in which the expression or activity of a product of an ASD-linked gene mutation is determined in a biological protein-containing sample obtained from the individual. Abnormal levels of the gene product or abnormal levels of the activity thereof, i.e. reduced or elevated levels, in comparison with levels that exist in healthy non-ASD individuals, are indicative of a risk of ASD, or may be indicative of ASD. Thus, a determination of the level and/or activity of the gene product of PTCHD1, may be used to determine the risk of ASD in an individual, or to diagnose ASD. Further, a determination of the level and/or activity of the gene product of PTCHD1AS1, PTCHD1AS2, and/or PTCHD1AS3 or splice variants thereof, may be used to determine the risk of ASD in an individual, or to diagnose ASD. As one of skill in the art will appreciate, standard assays may be used to identify and quantify the presence and/or activity of a selected gene product.

Embodiments of the invention are described by reference to the following specific exemplification which is not to be construed as limiting.

### EXEMPLIFICATION

### Methods

Subjects: CNVs at the PTCHD1 locus were initially assessed in 427 ASD patients as described (Marshall, C. R. et al., Am. J. Hum. Genet. 82, 477-488 (2008)). DNA samples from 900 individuals diagnosed with ASD were sequenced for PTCHD1 mutations, and compared to a reference nucleic acid sequence to identify mutations. In this regard, FIG. 1 illustrates the cDNA sequence (A) of the PTCHD1 gene and the corresponding amino acid sequence (B).

Among the samples assessed, 400 samples were collected at three sites, namely The Hospital for Sick Children (HSC) in Toronto and child diagnostic centers in Hamilton, Ontario and St, John's, Newfoundland. Details of these samples are published elsewhere (Moessner, R. et al., Am. J. Hum. Genet. 81, 1289-1297 (2007)). 420 ASD cases were recruited at Montreal, details of these samples are published elsewhere (Gauthier, J. et al., Mol. Psychiatry 11, 206-213 (2006)). Another 80 ASD probands from the Autism Genetic Resource Exchange (AGRE) were also included. The second cohort of 996 autism probands was recruited at different sites as a part of the Autism Genome Project (AGP); ascertainment is described elsewhere (Pinto, D. et al., Nature 466, 368-372 (2010)). 246 male patients with intellectual disability were recruited from the UK, United States, Australia, Europe and South Africa as the IGOLD study. A subset of 225 from this cohort were also used for sequence analysis of PTCHD1. Details of these samples are published elsewhere (Tarpey, P. S. et al., Nat. Genet. 41, 535-543 (2009)). 167 unrelated patients diagnosed with ADHD were recruited through the Department of Psychiatry at the Hospital for Sick Children, Toronto. Microarray data from controls included 1,123 (M=623, F=500) controls recruited from northern Germany as a part of the PopGen project, 1,234 (M=586, F=648) healthy controls of European origin recruited from the province of Ontario, Canada, 1,287 (M=383, F=904) controls from the Study of Addiction: Genetics and Environment (SAGE), 1,320 (M=589, F=1320) controls from Children's Hospital of Philadelphia (CHOP), 4783 (M=2460, F=2323) controls were recruited by the Wellcome Trust Case Control Consortium, 440 (M=158, F=282) controls were recruited by The Centre of Addiction and Mental Health (CAMH) and GlaxoSmithKline (GSK), and 59 (M=30, F=29) from the Centre d'Etude Polymorphisme Humaine (CEPH) HapMap controls (total N=5,023). More than 650 Ontario controls were obtained from The Centre for Applied Genomics (TCAG) and The Centre for Addiction and Mental Health (CAMH) and sequenced. Institutional ethical review board approval (CAMH, HSC, CHOP and all other collaborating institutions) was obtained for the study, and informed written consent was obtained for each family. Details of the clinical findings in families with PTCHD1 mutations or CNVs are summarized in Table 1.

TABLE 1
Clinical description of cases with disruptions at the PTCHD1 locus on Xp22.11

| | Genes; | # Chromosomes | | |
|---|---|---|---|---|
| Family ID | Mutation | Tested in Controls | Clinical Details in Proband‡ | Family Segregation Comments |
| Family 1 | PTCHD1, | 15,663 | Proband (deletion) = Autism (based on ADI & | Simplex family. |
| (1-0186) | PTCHD1AS2/3 | (M = 4,829 F = 10,834) | ADOS-Module 1) & ADHD. | Proband's brother DZ twin (deletion) = |
| | 167 Kb del | | Leiter-R brief IQ: 97 (42%)†; PLS-3: 86 (18%); | ASD features and Learning Disability. |
| | | | VABS: COM = 88 (21%); DLS = 79 (8%), | WASI: Non-Verbal IQ = 67 (1%), |
| | | | SOC = 80 (9%), MOT = 75 (5%), ABC = | Verbal IQ = 86 (18%); VABS: COM = |
| | | | 74 (4%). | 84 (14%), DLS = 95 (37%), SOC = |
| | | | | 104 (61%), ABC = 92 (30%) |
| | | | | Proband's sister (heterozygous |
| | | | | deletion) = non-ASD |
| Family 3 | PTCHD1 | 1101 | Proband (mutation) = Autism | Simplex family. No other siblings. |
| (S01407) | I173V | (M = 613 F = 488) | (based on ADI & ADOS-Module 1). | |
| | | | Non-Verbal IQ = 95, Verbal IQ = 85. | |
| Family 4 | PTCHD1 | 1193* | Proband (mutation) = Autism (based on ADI & | Simplex family. No other siblings. |
| (S01433) | ML336-7II | (M = 643 F = 550) | ADOS-Module 1). Some traits were observed | |
| | | | that might be related to schizophrenia. | |
| Family 5 | PTCHD1 | 869 | Proband (mutation) = High Functioning Autism | Simplex family. |
| (S01355) | E479G | (M = 531 F = 338) | | Proband's brother (no genotype data) = |
| | | | | non-ASD |
| Family 6 | PTCHD1 | 869 | Proband (mutation) = Autism | Multiplex family. |
| (AU0501) | L73F | (M = 531 F = 338) | | Proband's brother #1 (no mutation) = |
| | | | | ASD Proband's brother #2 |
| | | | | (mutation) = phenotype is currently |
| | | | | unclear. |
| Family 9 | PTCHD1 I173V | 1101 | Proband (mutation) = Autism (based on ADI & | Simplex family. |
| (1-0215) | and de novo | (M = 613 F = 488) | ADOS-Module 1), intellectual disability, | Proband's sister (mutation) = non-ASD |
| | ~1.1 Mb loss at | | hyperactive, poor motor coordination. Leiter-R | |
| | DPYD | | Brief IQ = 38. OWLS = 40 (<1%). VABS: | |
| | | | COM = 36(<1%); DLS = <20 (<1%), SOC = 31 | |
| | | | (<1%), ABC = 26 (<1%). | |
| Family 10 | PTCHD1 | 1101 | Proband (mutation) = Autism (based on ADI & | Simplex family. No other siblings |
| (3-0002) | V195I and 66 Kb | (M = 613 F = 488) | ADOS-Module 1). Severe expressive/receptive | |
| | de novo loss at | | language delay. CT head = Normal. | |
| | DPP6 | | | |
| Family 11 | PTCHD1AS1-3, | 15,663 | Proband (deletion) = Autism (based on ADI-R & | Simplex family. |
| (5298) | DDX53 | (M = 4,829 F = 10,834) | ADOS-Module 1), ID, speech delay, apraxia. Uses | Proband's sister (heterozygous |
| | 125 Kb del | | single words. Leiter Brief IQ: 42 (<1%). PPVT-4: | deletion) = non-ASD. |
| | | | 20 (<1%). VABS: COM = <20 (<1%); DLS = 47 | |
| | | | (<1%), SOC = 44 (<1%), ABC = 34 (<1%). | |
| Family 12 | PTCHD1AS1 | 15,663 | Proband (deletion) = Autism (based on ADI-R & | Mulitplex family. Paternal family |
| (5065) | 65 Kb del | (M = 4,829 F = 10,834) | ADOS-Module 4). Verbally fluent. | history of ASD. Proband's brother |
| | | | Leiter IQ: 71 (3%). VABS: COM = 68 (2%), | (no deletion) = Autism (based on ADI |
| | | | DLS = 45 (<1%), SOC = 58 (<1%), ABC = 52 | & ADOS-Module 4). Verbally Fluent. |
| | | | (<1%). | VABS: COM = 71 (3%), DLS = 38 |
| | | | | (<1%), SOC = 51 (<1%), ABC = 49 |
| | | | | (<1%). |
| Family 13 | 104 Kb del | 15,663 | Proband (deletion) = Autism (based on ADI & | Simplex family. |
| (3424) | | (M = 4,829 F = 10,834) | ADOS). WISC-R: Non-Verbal IQ = 58, Verbal | Proband's brother (no deletion) = |
| | | | IQ = 50, Total IQ = 50 | non-ASD |
| Family 14 | PTCHD1AS1 | 15,663 | Proband (deletion) = Autism (based on ADI-R & | Mulitplex family. Paternal family |
| (5111) | 59 Kb del | (M = 4,829 F = 10,834) | ADOS-Module 1). Uses single words. MRI = | history of ASD & ADHD. |
| | | | normal. Leiter IQ: 46 (<1%). VABS: COM = 37 | Proband's brother (no deletion) = |
| | | | (<1%), DLS = 31 (<1%), SOC = 52 (<1%), | Autism (based on ADI & ADOS- |
| | | | ABC = 37 (<1%). | Module 3). Verbally fluent. Leiter IQ: |
| | | | | 105 (63%). VABS: COM = 108 |
| | | | | (70%), DLS = 62 (1%), SOC = 92 |
| | | | | (30%), ABC = 83 (13%). Proband's |
| | | | | sister (heterozygous deletion) = non- |
| | | | | ASD, Bassen-Kornzweig syndrome. |
| | | | | Proband's father (no deletion) = non- |
| | | | | ASD, OCD. |
| Family 15 | PTCHD1AS1 | 15,663 | Proband (deletion) = Autism (based on ADI & | Multiplex family. |
| (3253) | 54 Kb del | (M = 4,829 F = 10,834) | ADOS) Non-Verbal IQ = 75, Verbal IQ = 56 | Proband's brother (no deletion) = ASD |
| | | | | Proband's sister (no deletion) = non- |
| | | | | ASD. |
| Family 16 | PTCHD1AS1-3, | 15,663 | Proband (deletion) = Autism (based on ADI & | Multiplex family. |
| (13047) | DDX53 | (M = 4,829 F = 10,834) | ADOS). No epilepsy, history of language delay | Proband's brother #1 (no deletion) = |
| | 389 Kb del | | followed by a rapid language learning progression. | Autism (based on ADI & ADOS), |
| | | | Average to above average Non-Verbal and Verbal | IQ = average to above average |
| | | | IQ. | Proband's brother #2 (no deletion) = |
| | | | | ASD Proband's sister (no CNV |
| | | | | data) = non-ASD, semantic-pragmatic |
| | | | | language disorder. |
| Family 17 | 101 Kb del | 15,663 | Proband (no deletion) = ASD | Multiplex family. |
| (8273) | | (M = 4,829 F = 10,834) | WISC III IQ: Non-verbal = 120, Verbal = 130 | Proband's brother (deletion) = ASD |
| | | | | Proband's sister #1 (deletion) = ASD |
| | | | | Proband's sister #2 (deletion) = ASD |
| Family 18 | PTCHD1AS1 | 15,663 | Proband (no deletion) = Autism (based on ADI & | Multiplex family. |
| (8013) | 65 Kb del | (M = 4,829 F = 10,834) | ADOS-Module 3). WISC-III: Non-Verbal IQ = | Proband's brother #1 (deletion) = |
| | | | 139 (>99%), Verbal IQ = 89 (23%). VABS: SOC = | Autism (based on ADI & ADOS- |
| | | | 76 (5%). | Module 3). WISC III: Total IQ = |
| | | | | 44 (1%). Proband's brother #2 |
| | | | | (deletion) = non-ASD. WPPSI-R: |
| | | | | Verbal IQ = 89 (23%), non-verbal = |
| | | | | 100 (50%). |
| Family 19 | PTCHD1AS1-3, | 15,663 | Proband (no deletion) = ASD | Multiplex family. |
| (3387) | DDX53 | (M = 4,829 F = 10,834) | | Proband's father (deletion) = Broad |
| | 213 Kb del | | | Autism Phenotype Proband's brother |
| | | | | (no deletion) = ASD Proband's sister |
| | | | | (deletion) = non-ASD. |
| Family 20 | PTCHD1AS1-3, | 15,663 | Proband (deletion) = ADHD, NVLD | Simplex family. |
| 1-27075 | DDX53 | (M = 4,829 F = 10,834) | Verbal IQ = 131, Performance IQ = 113. | Proband's sister #1 (genotype |
| | 388 Kb del | | Proband has some ASD spectrum features (disin- | unknown) = non-ASD Proband's |
| | | | terest in social relationships, preference for being | sister #2 (genotype unknown) = |
| | | | alone, difficulty with change and over-adherence | non-ASD |
| | | | to structure and rules, difficulty with reading non- | |
| | | | verbal cues resulting in social difficulties) but no | |
| | | | evidence of restricted, repetitive, or stereotyped | |
| | | | behaviour. | |

§All probands are male and are of European ancestry except for those in family 9 (Mixed European), family 4 (East Asian), and families 6 and 7 (Not available). The referring diagnosis for all probands is Autism Spectrum Disorder (ASD) except for Families 2, 7, 8 (intellectual disability; ID) and Family 20 (ADHD)
‡Abbreviations used: ADHD: Attention-Deficit Hyperactivity Disorder; BAP: Broad Autism Phenotype; NVLD: Non-verbal Learning Disability; ADOS: Autism Diagnostic Observation Schedule; ADI(-R): Autism Diagnostic Interview(-Revised); Leiter-R: Leiter International Performance Scale-Revised (non-verbal); WISC-(R or III): Wechsler Intelligence Scale for Children-(Revised or 3rd Edition); WPPSI-R: Wechsler Preschool and Primary Scale of Intelligence-Revised; VABS: Vineland Adaptive Behaviour Scale-consists of the following domains. COM—Communication, DLS—Daily Living Scales, SOC—Socialization, MOT—Motor Skills, ABC—Adaptive Behaviour Composite; PLS-3: Preschool Language Scale-3; OWLS: Oral and Written Language Scale; PPVT-4: Peabody Picture Vocabulary Test (4th Edition).
†Standard Score 100 ± 15(percentile)
*Controls included N = 92 of Asian ancestry

Copy Number Variation Analysis: Affymetrix 500K SNP arrays were used to assess CNVs in a cohort of 427 ASD cases. Details on the methods of copy number analysis and complete results are published elsewhere (Marshall, C. R. et al., Am. J. Hum. Genet. 82, 477-488 (2008)). Only the CNV result at PTCHD1 is described here. Another cohort of 996 autism probands was analyzed on 1M BeadChips (Illumina) (Pinto, D. et al., Nature 466, 368-372 (2010)). 246 male patients with ID were analyzed on a custom designed NimbleGen 385K array. Genomic DNA samples were sent to NimbleGen for the hybridizations to be performed. Each patient sample (Cy5-labelled) was co-hybridised with DNA from the reference sample NA10851 (Cy3-labelled; obtained from Coriell Cell Repository). After data normalisation, the ADM-1 algorithm (CGH Analytics 3.4, Agilent) was used for CNV discovery. The ADHD cohort was analyzed on Affymetrix 6.0 arrays. Three algorithms (Birdsuite, iPattern and Affymetrix Genotyping console (GTC)) were used to infer CNVs. The CEPH, PopGen and Ontario controls were analyzed on Affymetrix 6.0 arrays, SAGE controls were analyzed using 1M BeadChips (Illumina), and Illumina 550K arrays were used for the CHOP and CAMH\GSK controls. Similar methods were used to infer CNVs in controls. Fisher's Exact Test was used to calculate the two-tailed p value.

DNA Sequencing and Mutation Screening: PCR primers were designed with Primer 3 (v. 0.3.0) to amplify all three exons and intron-exon boundaries. PCR were performed under standard conditions, and products were purified and sequenced directly with the BigDye Terminator v3.1 Cycle Sequencing Ready Reaction Kit (Applied Biosystems).

X-Inactivation Studies: X Chromosome Inactivation assays were performed on genomic DNA extracted from peripheral blood as described (Allen, R. C. et al., Am. J. Hum. Genet. 51, 1229-1239 (1992)). Briefly, X Chromosome Inactivation was measured by the analysis of the (CAG)n repeat in the androgen receptor gene at Xq11-q12 before and after digestion with methylation sensitive restriction enzymes HhaI and HpaII. Quantitative PCR amplification of androgen receptor gene repeat alleles was compared, with and without restriction digestion, to determine the ratio of X-active/inactive alleles.

Expression Analysis and Protein Localization: Expression analysis and tissue distribution for PTCHD1, PTCHD1AS1 and PTCHD1AS2 was performed by RT-PCR, with a multiple tissue panel of first strand cDNA. The housekeeping gene G3PDH was used as a control. Origene human adult brain tissue panel was used to check the expression of PTCHD mRNA in different regions of the brain. qRT-PCR was performed with TaqMan Gene Expression assay Hs00288486, and samples were pre-normalized to GAPDH expression. Northern blot analysis was performed with a six tissue mRNA blot (BioChain). The BioChain FastHyb solution was used to hybridize the probe according to manufacturer's instructions. RNA in situ hybridization was performed on paraffin sections and whole-mounted fetal mouse and adult mouse brain using a 411 bp (chrX:152,008,934-152,009,344, UCSC Mouse July, 2007 (UCSC Genome Browser)) digoxigenin-labeled mouse antisense probe (and sense probe as negative control), using standard methods. To examine cellular localization of PTCHD1 protein, full-length human fetal brain PTCHD1 cDNA was PCR amplified and cloned into the pcDNA3.1/CT-GFP-TOPO expression vector (Invitrogen). After confirming sequence and orientation of the insert, COS-7 and SK-N-SH cells were transiently infected with 2 μg of purified construct DNA with SuperFect (Qiagen). 24 hours after transfection, the PTCHD1-GFP fusion protein was visualized in transfected cells using a Zeiss Axioplan 2 imaging microscope, equipped with the LSM510 array confocal laser scanning system, and the Zeiss LSM510 version 3.2 SP2 software package.

Luciferase Assays: A luciferase assay was performed to compare the effect of PTCH1, PTCH2 and PTCHD1 on Gli-dependent transcription with a previously described method (Nieuwenhuis, E. et al., Mol. Cell Biol. 26, 6609-6622 (2006)). Briefly, the 10T1/2 cells were transiently transfected with mixtures containing 0.1 μg β-galactosidase to normalize for transfection efficiency, 1 μg reporter plasmid (8× Glipro) encoding multimerized Gli binding sites fused to the luciferase gene and up to 1 μg of Gli2, PTCH1 or PTCH2 or PTCHD1. Gli-dependent transcription was measured and normalized by β-galactosidase. Data were replicated in independent experiments performed in triplicates. In another assay, 10T1/2 cells were transiently transfected with mixtures containing 0.1 μg β-galactosidase, 1 μg 8× Glipro reporter plasmid and purmorphamine, PTCH1 or PTCH2 or PTCHD1. The effect of PTCH1, PTCH2 and PTCHD1 on the endogenous Gli-dependent transcription was measured. Statistical significance was calculated asp below 0.05, using the Student's t-test.

Cytogenetic and CNV analysis of proband from Family 9: Localization of translocation breakpoints was performed by fluorescence in situ hybridization (FISH; performed in accordance with standard procedures) initially using bacterial artificial chromosome (BAC) clones across the suspected breakpoint regions, and then narrowing the search using fosmid clones. BAC clones were obtained from the RP11 human genomic library, and fosmid clones from the Whitehead fosmid library WIBR2. For the chromosome 19 locus, the clone G248P85500F11 was translocated, and thus distal to the breakpoint, while clone G248P85559B4 was not translocated, and thus proximal to the breakpoint. The breakpoint therefore lies within a 32 Kb region between these two clones (UCSC March 2006: Chr19: 7,843,511-7,874,724. This region encompasses just two genes: FLJ22184, LRRC8E. At the chromosome 21 translocation site, fosmid clone G248P87249E2 was translocated, and G248P89542E9 was not translocated, and the breakpoint thus lies within a ˜14.5 Kb region between these two clones, within an intron of the RUNX1 gene.

Whole-genome SNP analysis was performed using the Affymetrix 260K NspI SNP microarray. Analysis using the dCHIP and CNAG programs indicated a loss of heterozygosity from SNPs rs10875047 at Chr1:97,367,581 and rs822559 at Chr1:98,424,675 (inclusive; UCSC March 2006). This apparent deletion spans from intron 20 of the gene DPYD to include the first 20 DPYD exons, as well as two proximal putative genes, AK094607 and AX747691.

### Results

CNV Analysis of PTCHD1: Precise breakpoints of the 167 Kb deletion at PTCHD1 identified in the male proband from Family 1 were characterized. This CNV also disrupts long, spliced non-coding RNAs (ncRNAs) on the opposite strand that codes for PTCHD1, however, no other coding genes were interrupted. See FIG. 2 which depicts a detailed genomic organization of the PTCHD1 locus. Known genes, predicted CpG islands (>300 bp), predicted promoters (ElDorado Suite from Genomatix) and conserved sequences (>75% identity with chicken, >90% identity with opossum or 100% identity with dog or horse) are shown.

The 167 kb deletion was validated in the family using both PCR and SYBR-Green I-based real-time quantitative PCR (qPCR) and was found to be transmitted from a heterozygous unaffected mother to two affected dizygotic twin sons, also to an unaffected daughter (FIG. 3). X-chromosome inactivation (XCI) analysis of the mother, carrier of the PTCHD1 deletion, revealed a highly skewed allelic ratio of 94:6. The third male in Family 18 was assessed at age 4 and had speech and language problems, but was not available for further assessment. The father in Family 19 has a broader autism phenotype (BAP) (Pinto, D. et al., Nature 466, 368-372 (2010)). The proband in Family 20 (hatched) has ADHD plus BAP. A diamond symbol represents siblings who were not tested as part of the study, and with gender not indicated.

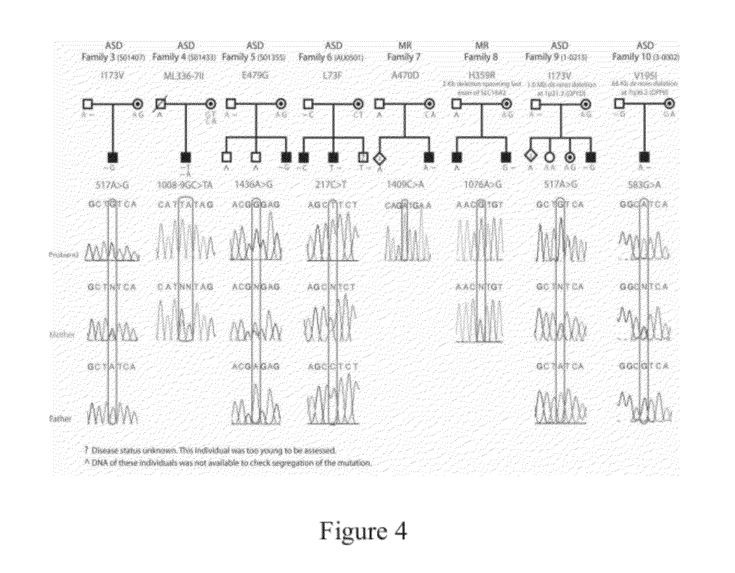

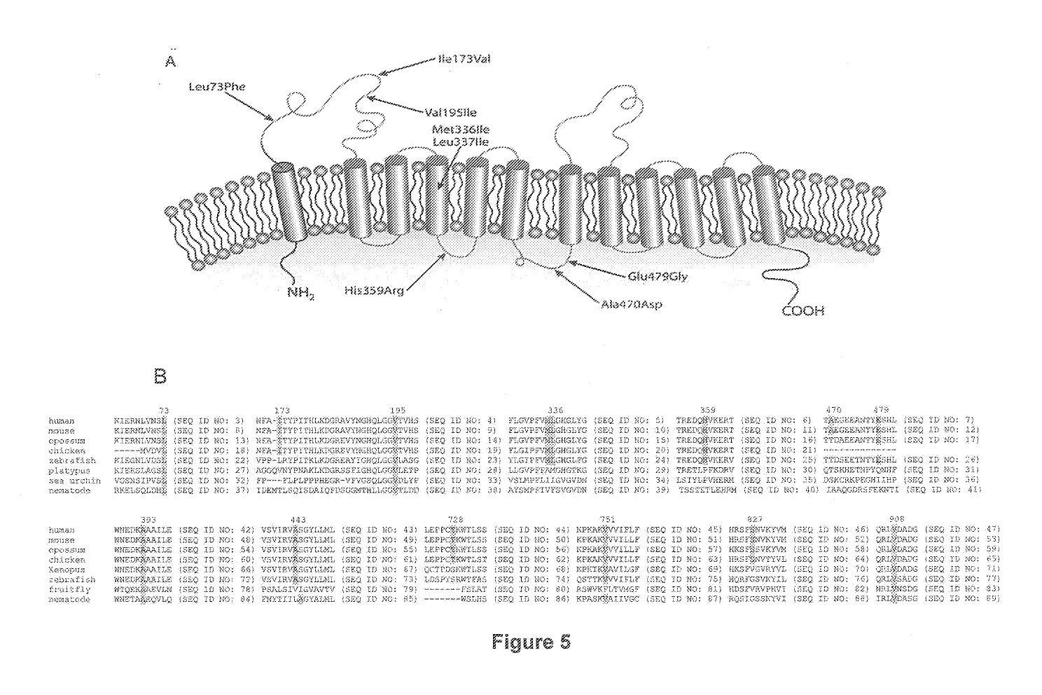

Mutation Screening of PTCHD1: In order to identify additional cases with PTCHD1 mutations, the coding regions in 900 (M=723; F=177) unrelated ASD cases and 225 unrelated male ID cases were sequenced. Missense changes were identified in unrelated ASD probands and ID probands (FIG. 3; FIG. 4; see also Table 1, above). In FIG. 5, the protein structure of the transmembrane protein PTCHD1 is illustrated. In 5A, twelve transmembrane domains (cylinders) and Patched-domain (line) were identified using the SMART tool (http://smart.embl-heidelberg.de/) with the Pfam domain option selected. In addition, the locations of missense sequence variants discovered among ASD and ID probands are shown. 5A shows the position of missense mutations among ASD and ID probands. Amino acid positions given are relative to the human PTCHD1 sequence (NP_{—}775766). Other sequences used include mouse (NP_{—}001087219), opossum (XP_{—}001366520), platypus (XP_{—}001512040), chicken (XP_{—}425565), zebrafish (XP_{—}690754), sea urchin (XP_{—}001199849) and nematode (C. elegans) (NP_{—}499380). 5B/C depicts PTCH1, showing missense mutations reported for holoprosencephaly₁₄₁₅, and includes sequences from human PTCH1 (NP_{—}000255), mouse (NP_{—}032983), opossum (XP_{—}001368370), chicken (NP_{—}990291), Xenopus laevis (NP_{—}001082082), zebrafish (XP_{—}001922161), fruitfly (NP_{—}523661) and nematode (C. elegans; NP_{—}495662).

All of these variants, which resulted in the substitution of highly conserved amino acids, were inherited from unaffected carrier mothers (FIG. 4). In six of the eight families the missense variants appear to segregate with the phenotype, however in Family 6 L73F did not segregate, (see FIG. 4 and Table 1 for details).

The entire coding region of PTCHD1 was sequenced in 700 control individuals (M=531 F=169), and none of the missense changes identified from among the ASD and ID patient cohorts has been detected. Only two missense changes have been identified: P252L from amongst the controls, and N497K reported in the SNP database (rs35880456, in 1 out of 39 screened; NCBI), both in females who were heterozygotes. Altogether, absence of PTCHD1 missense variants indicates that these variants are significantly enriched in the males with ASD (6/723 male ASD versus 0/531 male control: Fisher's exact test: p=0.042) and may contribute to the phenotype.

Additional controls were sequenced for the exons in which missense mutations were identified. Control chromosomes were tested for the sequence underlying the I173V and V1951 mutations (N=1101 chromosomes), the ML336_{—}337II mutation (N=1193), and the L73F and E479G mutations (N=869) and detected none of these variants.

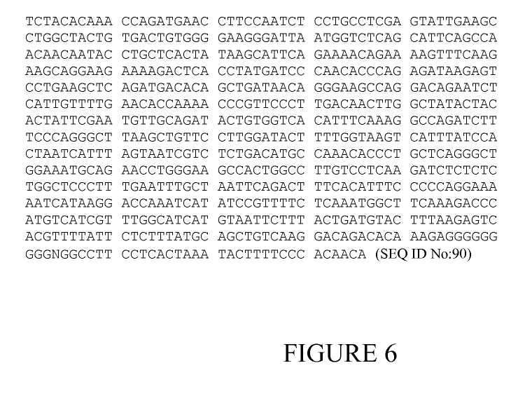

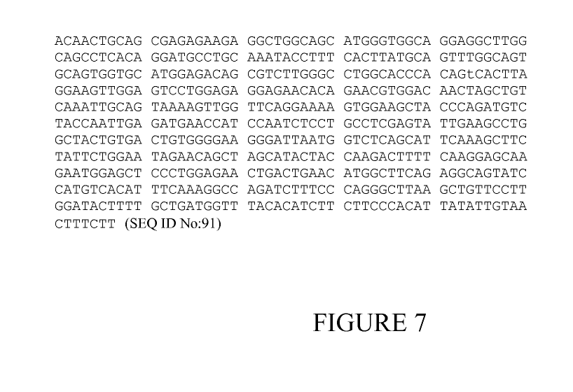

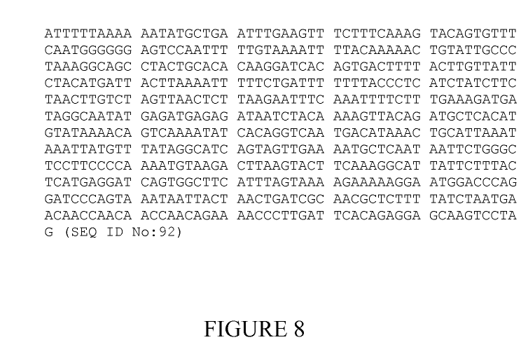

CNVs upstream of PTCHD1 (PTCHD1AS1/PTCHD1AS2 locus): Copy number variations were also identified upstream of the coding region for PTCHD1. A study of 996 ASD families examined with the Illumina 1M BeadChip (Pinto, D. et al., Nature 466, 368-372 (2010)) identified deletions in probands or affected siblings, and in a father with a diagnosis of Broad Autism Phenotype (BAP) (Hurley R. S. et al., J. Autism Dev. Disord. 37, 1679-1690 (2007); Constantino, J. N. et al., Biol. Psychiatry 57, 655-660 (2005)). All of the upstream CNVs occurred 5′ of PTCHD1, and overlapping with an anti-sense non-coding RNA, PTCHD1AS1/PTCHD1AS2. A tenth deletion at this upstream locus was identified in a patient from a CNV study of 167 unrelated attention deficit-hyperactivity disorder (ADHD) patients. The ADHD proband with the deletion also has a BAP diagnosis. See FIG. 2. Putative non-coding RNA transcripts PTCHD1AS1 (from cDNA clone IMAGE:1560626; BX115199) and PTCHD1AS2 (cDNA clone BRSTN2000219; DA355362) from human, mouse and rat genomes are also shown, with transcripts assembled from RT-PCR and 5′ RACE (PTCHD1AS3) results. The dotted line between the two exons in transcript PTCHD1AS1 indicates that this is a putative exon, identified through clone sequencing. This exon is putative because, although this location represents its best genomic hit, it only partially matches the 5′ end of the clone sequence. The consensus sequences for noncoding RNA of PTCHD1AS1, PTCHD1AS2 and PTCHD1AS3 are shown in FIGS. 6, 7 and 8, respectively.

In FIG. 2, Black boxes within the spliced transcripts indicate homologous exons between the sequences. White bars with black borders indicate CNV losses within this locus that have been identified in patients with ASD and controls. Cross-hatched or grey bars indicate CNV losses identified in patients with ADHD and ID, respectively. Lines within these bars indicate overlap with exons of known transcripts or ncRNA.

The breakpoints of the deletions for all families that are reported here were mapped by sequencing the junction. Breakpoints for all CNVs in controls were mapped by using the physical positions of microarray probe fragments. Deletions were validated with qPCR and exact breakpoints at the PTCHD1 locus were mapped (See Table 2). Additional CNV data for the individuals in other regions is included in Table 3.

TABLE 2
Breakpoint of deletions at the PTCHD1 locus:

| | | Deletion size | Method used to map |
|---|---|---|---|
| Family | Breakpoints* | (bp) | the breakpoints |
| Family 1 | chrX: 23,114,179- | 167,543 | Sequencing of |
| (5240) | 23,281,723 | | junction fragment. |
| Family 11 | chrX: 22,890,415- | 125,253 | Sequencing of |
| (5298) | 23,015,667 | | junction fragment. |
| Family 12 | chrX: 22,859,294- | 64,843 | Sequencing of |
| (5065) | 22,924,136 | | junction fragment. |
| Family 13 | chrX: 23,011,719- | 104,494 | Sequencing of |
| (3424) | 23,116,212 | | junction fragment. |
| Family 14 | chrX: 22,841,534- | 58,957 | Sequencing of |
| (5111) | 22,900,490 | | junction fragment. |
| Family 15 | chrX: 22,853,977- | 54,367 | Sequencing of |
| (3253) | 22,908,345 | | junction fragment. |
| Family 16 | chrX: 22,826,477- | 388,556 | Sequencing of |
| (13047) | 23,215,032 | | junction fragment. |
| Family 17 | chrX: 22,989,332- | 101,749 | Sequencing of |
| (8273) | 23,091,080 | | junction fragment. |
| Family 18 | chrX: 22,859,294- | 64,843 | Sequencing of |
| (8013) | 22,924,136 | | junction fragment. |
| Family 19 | chrX: 22,824,496- | 213,013 | Sequencing of |
| (3387) | 23,037,508 | | junction fragment. |
| Family 20 | chrX: 22,678,814- | 388,006 | Sequencing of |
| (1-27075) | 23,066,819 | | junction fragment. |

*refers to genome assembly HG18

TABLE 3
Additional CNVs in 9 subjects with upstream deletions:

| Family | Gender | Inheritance | Physical Position | Size (bp) | CNV | Cytoband | Genes |
|---|---|---|---|---|---|---|---|
| Family 1 | M | Maternal | 2:236932539_236990050 | 57,512 | 3 | 2q37.2 | IQCA1 |
| (5240) | | | | | | | |
| Family 11 | M | Paternal | 14:43889940_44003766 | 113,827 | 3 | 14q21.3 | No gene. |
| (5298) | | | | | | | |
| | M | Maternal | 16:16225138_16726778 | 501,641 | 3 | 16p12.3, | ABCC6, NOMO3 |
| | | | | | | 16p13.11 | |
| | M | | 16:18153166_18699648 | 546,483 | 3 | 16p12.3 | ABCC6P1, NOMO2, LOC |
| | | | | | | | 339047, RPS15A |
| Family 12 | M | Maternal | 1:17079505_17140083 | 60,579 | 1 | 1p36.13 | CROCC |
| (5065) | | | | | | | |
| | M | paternal | 3:1719782_1786952 | 67,171 | 3 | 3p26.3 | No gene. |
| | M | Maternal | 3:17494057_17542224 | 48,168 | 1 | 3p24.3 | TBC1D5 |
| | M | Maternal | 3:197219312_197527449 | 308,138 | 3 | 3q29 | PCYT1A, TCTEX1D2, TF |
| | | | | | | | RC, ZDHHC19, OSTalpha |
| | M | Maternal | 4:22488002_22620537 | 132,536 | 3 | 4p15.31 | No gene. |
| | M | Maternal | 10:68138586_68227559 | 88,974 | 1 | 10q21.3 | CTNNA3 |
| | M | paternal | 11:61516315_61632187 | 115,873 | 3 | 11q12.3 | No gene. |
| | M | Maternal | 16:21506626_21647775 | 141,150 | 3 | 16p12.2 | METTL9, IGSF6, OTOA |
| Family 13 | M | paternal | 5:98798044_98836932 | 38,889 | 1 | 5q21.1 | No gene. |
| (3424) | | | | | | | |
| | M | Maternal | 7:149089061_149159195 | 70,135 | 3 | 7q36.1 | SSPO, ZNF467 |
| Family 14 | M | Maternal | 18:66315754_66382003 | 66,250 | 1 | 18q22.2 | No gene. |
| (5111) | | | | | | | |
| Family 15 | M | NA | 5:20975886_21105120 | 129,235 | 1 | 5p14.3 | No gene. |
| (3253) | | | | | | | |
| | M | NA | 7:109552072_109593909 | 41,838 | 1 | 7q31.1 | No gene. |
| | M | NA | 9:11936421_12032535 | 96,115 | 1 | 9p23 | No gene. |
| Family 16 | M | Maternal | 1:244036261_245191978 | 1,160,000 | 1 | 1q44 | AHCTF1, TFB2M, LOC14 |
| (13047) | | | | | | | 9134, SCCPDH, SMYD3, |
| | | | | | | | C1orf71 |
| | M | Maternal | 9:24652558_24705098 | 52,541 | 1 | 9p21.3 | No gene. |
| | M | Maternal | 18:67894269_67931021 | 36,753 | 1 | 18q22.3 | No gene. |
| Family 17 | NA | | | | | | |
| (8273) | | | | | | | |
| Family 18 | NA | | | | | | |
| (8013) | | | | | | | |
| Family 19 | NA | | | | | | |
| (3387) | | | | | | | |
| Family 20 | NA | | | | | | |
| (1-27075) | | | | | | | |

SNP microarray data was analyzed from 10,246 control individuals (4,829 male; 5,417 female), for CNVs at PTCHD1 and the upstream region. In a 1.4-Mb region spanning from PTCHD1 to adjacent genes PRDX4 (proximal) and ZNF645 (proximal), 15 CNVs were identified (7 duplications and 8 deletions); however, it is notable that only 1 male control with a deletion was identified, which was 20.6 Kb in length and did not disrupt any known exons of any genes or non-coding RNAs, or any of the identified conserved or putative regulatory sequences. The remaining 7 deletions were all identified among female controls, consistent with the X-linked recessive inheritance observed for the PTCHD1 mutations. Thus, PTCHD1 and upstream deletions were not observed in 4,829 male controls, or in the Database of Genomic Variants (Iafrate, A. J. et al., Nat. Genet. 36, 949-951 (2004)), which suggests that the CNV directly disrupting PTCHD1 and the 6 CNVs located just upstream in unrelated ASD probands are associated with autism (male ASD cases N=7, out of 1,185; male controls N=0 out of 4,829; Fisher's exact test: p=1.2×10⁻⁵).

Expression and Functional Studies of PTCHD1: Expression analysis for the PTCHD1 and the ncRNA transcripts suggests that they are transcribed in brain regions, notably the cerebellum, as well as in other tissues (data not shown). RNA in situ hybridization of Ptchd1 in mouse showed widespread expression in the developing brain from E9.5/10.5 to P1 (data not shown), as well as broad expression in the adult mouse brain (6 months), with highest density in the cerebellum (see Allen brain atlas online (Allen Institute mouse brain atlas in situ hybridization data for Ptchd1: http://mouse.brain-map.org/brain/Ptchd1.html)).

Gene expression and genes co-expressed with PTCHD1 were also analyzed, from gene Affymetrix gene expression microarray analysis from BioGPS (Gene Atlas U133A, gcrma; http://biogps.gnf.org); UCLA Gene Expression Tool (UGET: http://genome.ucla.edu/˜jdong/GeneCorr.html; using human HG-U133_Plus_{—}2 microarrays (2), and correlation with mouse Ptchd1 using UGET and Mouse430_{—}2 microarrays. These algorithms correlate expression based on banked Affymetrix gene microarray data, and is not tissue specific. Ranking counts multiple probes as single hits, and excludes hypothetical proteins.PTCHD1 gene expression showed high correlation with expression of other cerebellar genes such as ZIC1, CADPS2, EN2, CBLN1, and with synaptic genes such as PCLO, NRXN3, SNAP25, SYT2, DPP6 and DPP10 (see Table 4).

To investigate its function, the sub-cellular localization of PTCHD1 was studied. It was found that a PTCHD1-GFP fusion protein predominantly localizes to the cell membrane (data not shown). It was further hypothesized that PTCHD1 may function in the Hh-signaling pathway and have similar functional attributes as PTCH1 and PTCH2. A Gli-dependent transcription assay was performed in Hh-responsive 10T1/2 cells to test whether PTCHD1 could interfere with Hh signaling. In 10T1/2 cells, overexpression of PTCH1 or PTCH2 inhibits transcription from a Gli-luciferase reporter containing multiple copies of the Gli protein-binding site in the presence of Smoothened agonist purmorphamine (Sinha, S. and J. K. Chen, Nat. Chem. Biol. 2, 29-30 (2006)) or Gli2 (data not shown). Similar to PTCH proteins, PTCHD1 also exerted a statistically significant inhibitory effect in these assays suggesting that PTCHD1 functions in the Hedgehog signalling pathway.

TABLE 4
Genes co-expressed with PTCHD1

| | | | | |
|---|---|---|---|---|
| | | | OMIM | |
| Gene Name | Correlation | Rank # | # | Comments |

A. BioGPS co-expression data for PTCHD1 from Gene Atlas, U133A

| | | | | |
|---|---|---|---|---|
| PTCHD1 | 1 | 1 | | |
| ZIC1 | 0.7564 | 2 | 600470 | Zinc finger protein in cerebellum; homologue of Gli |
| GABRD | 0.7064 | 12 | 137163 | Receptor subunit (delta) for GABA neurotransmitter |
| MAB21L1 | 0.6916 | 17 | 601280 | Autism susceptibility locus, AUTS3, candidate gene |
| CBLN1 | 0.6832 | 21 | 600432 | Precerebellin 1 |
| CADPS2 | 0.6827 | 22 | 609978 | Cerebellar gene; involved in vesicular trafficking; autism candidate gene |
| CACNA1A | 0.6801 | 23 | 601011 | Gene for spinocerebellar ataxia 6 |
| CALN1 | 0.6675 | 26 | 607176 | Calneurin 1; cerebellar homologue of calmodulin |
| NRXN3 | 0.6041 | 42 | 600567 | Neurexin 3; synaptic adhesion and presynaptic voltage-gated Cat²⁺ signalling |
| EN2 | 0.5799 | 50 | 131310 | Engrailed 2; candidate gene at autism locus, AUTS10 |
| SYT2 | 0.5782 | 51 | 600104 | Synaptotagmin 2; synaptic vesicle associated protein, CA²⁺ sensor |
| GRM1 | 0.5747 | 52 | 604473 | Metabotropic glutamate neurotransmitter receptor |
| GABRA6 | 0.5171 | 77 | 137143 | Receptor subunit (alpha-6) for GABA neurotransmitter |
| SNAP25 | 0.5034 | 87 | 600322 | Synaptosomal-associated protein |

B. UGET co-expression data for PTCHD1 from HG-U133_Plus_2 platform

| | | | | |
|---|---|---|---|---|
| PTCHD1 | 0.85455 | 1 | | |
| SNAP25 | 0.5389 | 7 | 600322 | Synaptosomal-associated protein |
| CACNA1A | 0.52815 | 10 | 601011 | Gene for spinocerebellar ataxia 6 |
| NRXN3 | 0.514 | 13 | 600567 | Neurexin 3; synaptic adhesion and presynaptic voltage-gated Cat²⁺ signalling |
| GABRA6 | 0.50935 | 15 | 137143 | Receptor subunit (alpha-6) for GABA neurotransmitter |
| GRM1 | 0.50555 | 19 | 604473 | Metabotropic glutamate neurotransmitter receptor |
| GABRD | 0.4958 | 24 | 137163 | Receptor subunit (delta) for GABA neurotransmitter |
| KCNC1 | 0.4935 | 25 | 176258 | Voltage-gated K+ channel, Shaw-related, Kv3.1 |
| SYT4 | 0.4934 | 26 | 600103 | Synaptotagmin 4; synaptic vesicle associated protein, CA²⁺ sensor |
| CBLN3 | 0.4867 | 32 | 612978 | Precerebellin 3 |
| DPP6 | 0.4771 | 45 | 126141 | Dipeptidyl peptidase 6: forms complex with Kv4.2 channels at synapse |
| CADPS2 | 0.4699 | 54 | 609978 | Cerebellar gene; involved in vesicular trafficking; autism candidate gene |

C. UGET co-expression data for mouse Ptchd1 from Mouse430_2 platform

| | | | | |
|---|---|---|---|---|
| Ptchd1 | 0.7053 | 1 | | |
| Olfm3 | 0.4714 | 2 | 607567 | Olfactomedin 3 |
| Gria4 | 0.4397 | 3 | 138246 | Glutamate receptor (AMPA); L-glutamate-gated ion channel |
| Pclo | 0.4235 | 5 | 604918 | Piccolo; presynaptic cytoskeletal matrix component |
| Dpp10 | 0.4165 | 9 | 608209 | Dipeptidyl peptidase 10; forms complex with Kv4.2 channels at synapse |
| Cadps2 | 0.39 | 19 | 609978 | Cerebellar gene; involved in vesicular trafficking; autism candidate gene |
| Nrxn3 | 0.3879 | 21 | 600567 | Neurexin 3; synaptic adhesion and presynaptic voltage-gated Cat²⁺ signalling |
| En2 | 0.3816 | 30 | 131310 | Engrailed 2; candidate gene at autism locus, AUTS10 |

Gene Affymetrix gene expression microarray analysis from A. BioGPS (Gene Atlas U133A, gcrma; http://biogps.gnf. org); B. UCLA Gene Expression Tool (UGET: http://genome.ucla.edu/~jdong/GeneCorr.html; using human HG-U133_Plus_2 microarrays (2), and C. correlation with mouse Ptchd1 using UGET and Mouse430_2 microarrays. These algorithms correlate expression based on banked Affymetrix gene microarray data, and is not tissue specific. Ranking counts multiple probes as single hits, and excludes hypothetical proteins.

RT-PCR failed to find evidence for a shortened 3′ PTCHD1 transcript from individual with PTCHD1 exon 1 deletion: It was speculated that the difference in phenotype between the PTCHD1 deletion families, could be explained by residual PTCHD1 protein function in relevant brain regions in Family 1 due to downstream transcription and translation of a shorter isoform, possibly driven by a secondary promoter just upstream of exon 2, resulting in the milder ASD symptoms, rather than the severer ID with the full deletion. However, RT-PCR did not detect any evidence of shorter downstream transcripts.

RT-PCR and 5′ RACE (Rapid Amplification of cDNA Ends) analysis of the ncRNAs, PTCHD1AS1 and PTCHD1AS2 and the PTCHD1 gene: By RT-PCR, the annotated exons of PTCHD1AS1 and PTCHD1AS2 were amplified from human cerebellum cDNA. Sequencing of RT-PCR product confirmed the current annotation of the ncRNAs. Additionally, the annotation of PTCHD1AS1 was verified by re-sequencing of the IMAGE clone 1560626.

It was attempted to identify additional 5′ sequence of the ncRNAs and PTCHD1 by 5′ RACE analysis using the Clontech Marathon-Ready™ fetal brain cDNA (Cat. No. 639300). According to the manufacturer instructions the gene specific primers were designed for PTCHD1AS1, PTCHD1AS2 and PTCHD1 and RT-PCR was performed. The PCR products were cloned into the Promega pGEM®-T Easy Vector and the clones were sequenced using standard methods. No additional upstream sequence for PTCHD1 could be found; however, for the PTCHD1AS1 at least two additional exons were identified. One of these exons completely overlaps with the PTCHD1AS2 exon 2 (chrX:23,198,089-23,198,215), while the second exon mapped further upstream at chrX:23,261,313-23,261,767 (UCSC 2006). RT-PCR also identified another splice variant with an initial exon at ChrX:23,262,967-23,262,009, which skips to exon 2 in the current annotation of PTCHD1AS1. It is possible that the extremely GC-rich nature of the 5′ region of PTCHD1 prevented the finding of additional upstream sequence.

Alternative 5′ exons for PTCHD1AS1, identified by 5′RACE, are shown in Table 5 below.

TABLE 5
Alternative 5′ exons for PTCHD1AS1, identified by 5′RACE

| | | Size | | |
|---|---|---|---|---|
| NCRNA | Exon | (bp) | Coordinates | Comments |
| PTCHD1AS1 | 1^{I} | 126 | chrX: | This exon is alternatively |
| | | | 23,198,089- | spliced and completely |
| | | | 23,198,214 | overlaps with the exon 2 |
| | | | | of the NCRNA355362. |
| PTCHD1AS1 | 1^{II} | 455 | chrX: | Starts 1.1 Kb upstream of |
| | | | 23,261,313- | PTCHD1 and overlaps with |
| | | | 23,261,767 | the exon 1 of mouse transcript |
| | | | | AK028243 and the PTCHD1 |
| | | | | CpG island. |
| PTCHD1AS1 | 1^{III} | 43 | chrX: | Starts ~900 bp upstream of |
| | | | 23,261,967- | PTCHD1 and overlaps with |
| | | | 23,262,009 | the PTCHD1 CpG island. The |
| | | | | transcript starting from this |
| | | | | exon skips the Exon 1^{II}, 1^{I} and |
| | | | | exon 1. |

The relevant sequences are as follows:

Sequence of exon 1^{I:}
(SEQ ID No: 14)
CAATTGGTAGACATCTGGGTAGCTTCCACTTTTCCTGAACCAACTTTTAC
TGCAATTTGACAGCTAGTTGTCCACGTTCTGTGTTCTCCTCTCCAGGACT
CCAACTTCCTAAGTGGCTGTGGGTGC
Sequence of exon 1^{II;}
(SEQ ID No: 15)
ACCTGTGCGTGGCCGTTCCCGCCGCCGCCGCAGGTCTATCCCGGGGCCGA
AGCCGGCGCCCGCCTTCTCGGGGAATTCTCCGGAGGGGGAGTGCGAGGGG
AACCACGGTGACTGCCTGCTAGCTCACGGCTGGCGCGCACACGCACACGC
CCAACTTTGCCAAGCCGTCGGCGCCCCGCGGGCTCCCCCGCGCCCCCTGC
GGCTCAACACGCTCGGAGACCTGTATCTCTCCTGCTCTGAGATAAGGTTC
CCTCCACTCTCACACCTTCGCATGTAGGGGAGGAGAGGGCGGAGTGAGGC
AGAGAAGGGGGTTAATGCTACTGACTCCCTGGCCAGCCTTTCTCAAACAC
TCTACGCCCGCAGGGGCGCCCGCGCCAGCCACGCCGCACCAGGTCCCCCA
GACCTGCTGGTGACGACAGAGAGAGGAGGAGGAAGAGAAGGCAGGGCGAA
GAACC
Sequence of exon 1^{III:}
(SEQ ID No: 16)
CTTTTGAGTGGACGTGCTCCAGACACACACCCGGACCCCGTGG

Putative promoter and enhancer sequences in intergenic region between DDX53 and PTCHD1: The identification of predicted promoter sequences may indicate the presence of an alternative upstream transcription start site for PTCHD1 (or possibly another unknown gene), that may be disrupted by the CNVs identified upstream of PTCHD1 in ASD families (see supra). The Genomatix ElDorado suite was used to predict promoter sequences. The promoter sequence for DDX53 is (hg18/UCSC March 2006 build):

(SEQ ID No: 17)
TCTACACAAACCAGATGAACCNTCCAATCTCCTGCCTCGAGTATTGAAGCCTGGCTACTGTGACTGTGGG
GAAGGGATTAATGGTCTCAGCATTCAGCCAACAACAATACCTGCTCACTATAAGCATTCAGAAAACAGAA
AAGTTTCAAGAAGCAGGAAGAAAAGACTCACCTATGATCCCAACACCCAGAGATAAGAGTCCTGAAGCTC
AGATGACACAGCTGATAACAGGGAAGCCAGGACAGAATCTCATTGTTTTGAACACCAAAACCCGTTCCCT
TGACAACTTGGCTATACTACACTATTCGAATGTTGCAGATACTGTGGTCACATTTCAAAGGCCAGATCTT
TCCCAGGGCTTAAGCTGTTCCTTGGATACTTTTGGTAAGTCATTTATCCACTAATCATTTAGTAATCGTC
TCTGACATGCCAAACACCCTGCTCAGGGCTGGAAATGCAGAACCTGGGAAGCCACTGGCCTTGTCCTCAA
GATCTCTCTCTGGCTCCCTTTGAATTTGCTAATTCAGACTTTCACATTTCCCCCAGGAAAAATCATAAGG
ACCAAATCATATCCGTTTTCTCAAATGGCTTCAAAGACCCATGTCATCGTTTGGCATCATGTAATTCTTT
ACTGATGTACTTTAAGAGTCACGTTTTATTCTCTTTATGCAGCTGTCAAGGACAGACACAAAGAGGGGGG
GGGNGGNCTTCCTCACTAAATACTTTTCCCACAACA

In addition to promoter sequences at the 5′ ends of DDX53 and PTCHD1, on the plus strand a putative promoter sequence was identified in the intergenic region, from ChrX:22,927,508-22,928,108 (hg18/UCSC March 2006 build):

(SEQ ID No: 18)
AATGATGAATTTATCCTGACAAAGTACTGTATTCACTCCAAAAGAAATTT
ACCAAAATAAATGAACACACGAATATATAAATAAATAGTTTTACTTTAAA
TGCATTATTTTTTTCTCTTAGGGAAATAACTGGCTTATATAAAGGACAAT
GTGTATATGGTGTGTATGTTTAAGGCGTGCTTCAAGGTTGCTCTCAAGCT
GAGCCAGAACTATCACGAGAAGAGTGAAAGGAGCACCCGGGACGCAGAAG
TTAAGGAGGCAGTTACTCCTAGGGTCCTGTAAGTGCTGGCAGGGTCAGCC
CGTGAGAGTGAGTGCCTCTTTAAATTTGCGTCACAGACGCCTGCTTACCT
CACCCCAGTCCAAGCCCTGTGATTGGTCAGGCCATCAAAGCCTCGCCCCC
TACACGACCCGGAATTCGACGCCAACACTGGTTTCTGGGGCAACTTCTGC
GTAGCTATGTGACTAGCACCCGGAAATAATTGCCACCGCCATCTTTTGGT
GCAGAAGGTGACGGGAAACAGGCCGCAGACCTGAACTTCCAACCGTATGT
AGGCGAGAAGCCGGTGCCGATACTCCCACTATCCCACAATGTCCCACTGG
G

This putative promoter lies ahead of ENSEMBL predicted non-coding transcript ENST00000407873. On the minus strand a putative promoter sequence was identified in the intergenic region, from ChrX: chrX:23,022,123-23,022,723, which lies just ahead of ENSEMBL predicted non-coding transcript ENST00000356867 and an EST clone (AU118198) (hg18/UCSC March 2006 build):

(SEQ ID No: 19)
ATTTTTAAAAAATATGCTGAATTTGAAGTTTCTTTCAAAGTACAGTGTTT
CAATGGGGGGAGTCCAATTTTTGTAAAATTTTACAAAAACTGTATTGCCC
TAAAGGCAGCCTACTGCACACAAGGATCACAGTGACTTTTACTTGTTATT
CTACATGATTACTTAAAATTTTTCTGATTTTTTTACCCTCATCTATCTTC
TAACTTGTCTAGTTAACTCTTAAGAATTTCAAATTTTCTTTGAAAGATGA
TAGGCAATATGAGATGAGAGATAATCTACAAAAGTTACAGATGCTCACAT
GTATAAAACAGTCAAAATATCACAGGTCAATGACATAAACTGCATTAAAT
AAATTATGTTTATAGGCATCAGTAGTTGAAAATGCTCAATAATTCTGGGC
TCCTTCCCCAAAATGTAAGACTTAAGTACTTCAAAGGCATTATTCTTTAC
TCATGAGGATCAGTGGCTTCATTTAGTAAAAGAAAAAGGAATGGACCCAG
GATCCCAGTAAATAATTACTAACTGATCGCAACGCTCTTTTATCTAATGA
ACAACCAACAACCAACAGAAAACCCTTGATTCACAGAGGAGCAAGTCCTA
G

The ElDorado Suite from Genomatix, as well as the FPROM algorithm from the Softberry suite, was also used to predict promoter/enhancer sequences just upstream of the FAM3C2 predicted pseudogene.

Comparative sequence analysis indicated a number of regions located in the gene desert upstream of PTCHD1 and between DDX53 where nucleotide sequence conservation is relatively high through vertebrate evolution or through mammalian evolution. Such conserved regions may represent functional regions, possibly cis-regulatory sequences for PTCHD1. Regions were selected through the Vertebrate Multiz Alignment & PhastCons Conservation (28 Species) track on the UCSC (March 2006 build) browser. Results are shown in Table 1 and indicate which conserved elements overlap with CNV losses upstream of PTCHD1.

eQTL at PTCHD1 locus: The SNP rs7878766, located within PTCHD1 intron 1, has been reported as a quantitative trait locus for expression of mRNA levels of MAP8KIP2 in control brain cortex (http//eqtl.uchicago.edu), with a QTL score of 5.3. RefSeq Summary reports this to encode a scaffold protein involved in the c-Jun N-terminal kinase signaling pathway, and is thus thought to act as a regulator of signal transduction. Using mRNA by SNP Browser 1.0.1, other SNPs at the PTCHD1 locus that showed as suggestive QTLs for mRNAs included rs5925800 (ACSM2A; LOD=5.039, p=1.5×10⁻⁶; GALNT4, LOD=5.095, p=1.3×10⁻⁶; PIK3C2G, LOD=5.27, p=8.4×10⁻⁷), rs868659 (DLEU2, LOD=5.427, p=5.8×10⁻⁷), and rs6526278 (SGCG, LOD=5.248, p=8.8×10⁻⁷).

In summary, the data indicate that mutations at the PTCHD1 locus are highly penetrant and strongly associated with ASD (including BAP) and ID in ˜1.1% and ˜1.3% of the individuals analyzed, respectively (based on probands for whom comprehensive mutation screening, for both CNVs and sequence variants, has been performed (4 out of 353 ASD, and 3 out of 225 ID). As one of skill in the art will appreciate, mutations indicative of ASD and ID may vary from the exact CNVs identified (e.g. in Table 2 or other mutations), but will include at least a portion of one or more of the identified CNVs.

Overall, the findings are reminiscent of genetic findings for several other X chromosome genes, including NLGN4 (Jamain, S. et al., Nat. Genet. 34, 27-29 (2003); Laumonnier, F. et al., Am. J. Hum. Genet. 74, 552-557 (2004)) and IL1RAPL1 (Bhat, S. S. et al., Clin. Genet. 73, 94-96 (2008); Piton, A. et al., Hum. Mol. Genet. 17, 3965-3974 (2008); Carrie, A. et al., Nat. Genet. 23, 25-31 (1999)), in that mutations can apparently cause either ASD or ID (or both), and thus PTCHD1 may be a gene for both. IL1RAPL1, for example, was initially reported as a gene for non-syndromic X-linked ID (Carrie, A. et al., Nat. Genet. 23, 25-31 (1999)), and then subsequently was also found to harbor mutations in ASD pedigrees (Bhat, S. S. et al., Clin. Genet. 73, 94-96 (2008); Piton, A. et al., Hum. Mol. Genet. 17, 3965-3974 (2008)). Families have also been identified in whom at least two loci may be contributing to the pathogenesis of ASD, and other families bearing upstream microdeletions that disrupt a complex non-coding RNA, providing possible genetic explanations for the clinical heterogeneity of these disorders. Finally, the results raise the possibility that Hh signaling may be perturbed in these conditions.

What is claimed is:

1. A method of determining the risk of ASD in an individual comprising:
analyzing a nucleic acid-containing sample obtained from the individual for the presence or absence of a genomic sequence mutation at the PTCHD1 locus wherein the mutation comprises a deletion of a region upstream to the PTCHD1 gene, a disruption of a non-coding RNA selected from PTCHD1AS1, PTCHD1AS2, or PTCHD1AS3, or splice variants of these ncRNAs, or a disruption of other regulatory elements upstream of the PTCHD1 coding region, and wherein the presence of the mutation is indicative of a risk of ASD.

2. The method as defined in claim 1, wherein the mutation comprises a deletion of a region upstream to the PTCHD1 gene.

3. The method as defined in claim 2, wherein the deletion comprises at least a portion of a region of the X chromosome selected from the regions: 23,114,179-23,281,723, 22,890,415-23,015,667, 22,859,294-22,924,136, 22,859,294-22,924,136, 22,841,534-22,900,490, 22,853,977-22,908,345, 22,826,477-23,215,032, 22,989,332-23,091,080, 22,859,294-22,924,136, 22,824,496-23,037,508 and 22,678,814-23,066,819.

4. The method as defined in claim 1, wherein the mutation comprises a disruption of a non-coding RNA selected from PTCHD1AS1, PTCHD1AS2, or PTCHD1AS3, or splice variants of these ncRNAs.

5. The method as defined in claim 4, wherein the mutation comprises a disruption of a non-coding RNA PTCHD1AS1, or splice variants thereof

6. The method as defined in claim 4, wherein the mutation comprises a disruption of a non-coding RNA PTCHD1AS2 or a splice variant thereof.

7. The method as defined in claim 4, wherein the mutation comprises a disruption of a non-coding RNA PTCHD1AS3 or a splice variant thereof.

8. The method as defined in claim 1, wherein the mutation comprises a disruption of regulatory elements upstream of the PTCHD1 coding region.

9. The method of claim 8, wherein the mutation comprises a disruption of at least a portion of a promoter sequence in the intergenic region, from ChrX:22,927,508-22,928,108 or a promoter sequence in the intergenic region, from ChrX: chrX:23,022,123-23,022,723.

10. The method of claim 8, wherein the mutation comprises a disruption of cis-regulatory sequences for PTCHD1.
